(19) United States
(12) Patent Application Publication
(10) Pub. No.: US 2023/0203025 A1
(43) Pub. Date: Jun. 29, 2023

(54) ENHANCERS OF PARTICULATE GUANYLYL CYCLASE RECEPTOR A

(71) Applicants: Mayo Foundation for Medical Education and Research, Rochester, MN (US); Sanford Burnham Prebys Medical Discovery Institute, La Jolla, CA (US)

(72) Inventors: John C. Burnett, Jr., Rochester, MN (US); Jeson Sangaralingham, Rochester, MN (US); Siobhan Malany, Orlando, FL (US); Satyamaheshwar Peddibhotla, Orlando, FL (US); Paul Mitchell Hershberger, Satellite Beach, FL (US); Patrick Maloney, Orlando, FL (US); Edward Hampton Sessions, Jr., Orlando, FL (US)

(21) Appl. No.: 17/928,468

(22) PCT Filed: May 28, 2021
(86) PCT No.: PCT/US2021/034889

§ 371 Date: Nov. 29, 2022

Related U.S. Application Data
(60) Provisional application No. 63/032,330, filed on May 29, 2020.

(51) Int. Cl. C07D 417/12; A61P 9/12

(52) U.S. Cl. CPC C07D 417/12; A61P 9/12

(57) ABSTRACT

In some embodiments, the present disclosure provides a compound of Formula (I), as described herein, or a pharmaceutically acceptable salt thereof. Pharmaceutical compositions comprising the compound of Formula (I), and methods of treating, e.g., metabolic diseases using the compound of Formula (I) are also provided.

### CLAIM OF PRIORITY

This application claims priority to U.S. Provisional Patent Application Ser. No. 63/032,330, filed on May 29, 2020, the entire contents of which are hereby incorporated by reference.

### FEDERALLY SPONSORED RESEARCH AND DEVELOPMENT

This invention was made with government support under DK103850 awarded by the National Institutes of Health. The government has certain rights in the invention.

### TECHNICAL FIELD

This invention relates to organic compounds, and more particularly to 4-halobenzo[d]thiazole compounds useful in treating various conditions such as cardiovascular, renal, and metabolic diseases, as well as cancer.

### BACKGROUND

Metabolic disease continues to grow worldwide, representing one of the greatest burdens in human health. Metabolic disease, often referred to as metabolic syndrome, encompasses obesity, type 2 diabetes (T2DM), insulin resistance, hyperlipidemia and hypertension, and represents a global challenge to human health.

Cardiovascular disease (CVD), including myocardial infarction, stroke, and hypertension, also presents a significant socioeconomic burden. CVD remains the leading cause of death in the United States. The rates of CVD mortality per 100,000 people are currently nearly 400 for women, and nearly 700 for men.

Likewise, renal (kidney) disease is associated with a tremendous economic burden. High-income countries typically spend more than 2-3% of their annual health-care budget on the treatment of end-stage kidney disease, even though those receiving such treatment represent under 0.03% of the total population.

Finally, cancer is one of the leading causes of death in contemporary society. The numbers of new cancer cases and deaths is increasing each year. Currently, cancer incidence is nearly 450 cases of cancer per 100,000 men and women per year, while cancer mortality is nearly 71 cancer deaths per 100,000 men and women per year.

### SUMMARY

Atrial (ANP) and B-type natriuretic peptide (BNP) bind to the particulate guanylyl cyclase receptor A (pGC-A) that is highly expressed in heart, kidney, adrenals, vasculature and adipocytes. Following pGC-A activation, the second messenger 3′, 5′ cyclic guanosine monophosphate (cGMP) is produced resulting in widespread actions, including blood pressure lowering, renal enhancing, cardioprotective, and renin-angiotensin-aldosterone system (RAAS) suppressing properties. Advantageous metabolic actions of pGC-A include lipolysis, browning of adipocytes, stimulation of skeletal muscle energetics and release of adipokines such as adiponectin. The present disclosure is based, at least in part, on the realization that 4-halobenzo[d]thiazole compounds are positive allosteric modulators of pGC-A, and, therefore, are useful in treating cardiovascular, renal, and metabolic diseases. As a further advantage, the compounds of the present disclosure are orally bioavailable.

In a first general aspect, the present disclosure provides a compound of Formula (I):

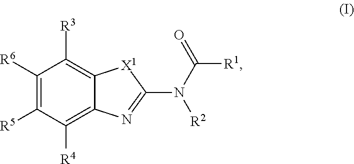

or a pharmaceutically acceptable salt thereof, wherein X¹, R¹, R², R³, R⁴, R⁵, and R⁶ are as described herein.

In a second general aspect, the present disclosure provides a pharmaceutical composition comprising the compound of Formula (I), or a pharmaceutically acceptable salt thereof, and a pharmaceutically acceptable carrier.

In a third general aspect, the present disclosure provides a method of modulating particulate guanylyl cyclase receptor A (pGC-A) in a cell, the method comprising contacting the cell with an effective amount of the compound of Formula (I), or a pharmaceutically acceptable salt thereof.

In a forth general aspect, the present disclosure provides a method of modulating particulate guanylyl cyclase receptor A (pGC-A) in a subject, the method comprising administering to the subject in need thereof an effective amount of the compound of Formula (I), or a pharmaceutically acceptable salt thereof, or the pharmaceutical composition comprising same.

In a fifth general aspect, the present disclosure provides a method of treating or preventing a disease or condition responsive to modulation of a particulate guanylyl cyclase receptor A (pGC-A) in a subject, the method comprising administering to the subject in need thereof a therapeutically effective amount of the compound of Formula (I), or a pharmaceutically acceptable salt thereof, or the pharmaceutical composition comprising same. In some embodiments, the disease or condition is selected from metabolic disease, cardiovascular disease, and kidney disease. Suitable examples of these diseases are described herein.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the present application will be apparent from the following detailed description and figures, and from the claims.

### DESCRIPTION OF DRAWINGS

FIG. 1 is a schematic representation showing pGC-A receptor, to which ANP and BNP bind, possesses pleiotropic actions via cGMP generation that leads to a therapeutic effect for cardiovascular, renal and metabolic disease, as well as cancer.

FIG. 2 is a line plot showing dose-dependent activity (via cGMP generation) of compound 1 (Table 1) in primary (HEK pGC-A) and secondary (HEK pGC-B) assays compared to compound A (Example 1).

FIG. 3 contains a table showing EC50 cGMP values for Compounds A and 1 in the primary HEK pGC-A and pGC-B selectivity and counterscreen (HEK parental) assays and in vitro solubility and stability parameters.

FIG. 4 contains line plot showing concentration-response curves for cGMP response of compound 19 in pGC-A expressing cells in the presence and absence of ANP and cGMP response of compound 19 in pGC-B expressing cells in the presence of CNP.

FIG. 5 is an SPR sensorgram showing the binding of compound 19 from 2.5 to 20 μM concentrations with 0.25 μM of the pGC-A receptor extracellular domain. K_{D}=4.1 μM.

FIG. 6 is a line plot showing plasma concentration of compound 19 after intravenous dosing at 5 mg/kg.

FIG. 7 is a line plot showing plasma concentration of compound 19 after oral dosing at 10 mg/kg.

FIG. 8 is a line plot showing plasma concentration of compound 19 after intravenous and oral dosing.

FIG. 9 contains a bar graph showing generation of cGMP in human cardiomyocytes stimulated by ANP (10⁻¹⁰ M) in absence (Veh) or presence of 1, 5 or 10 μM of compound 19. *P<0.05 vs. Veh.

FIG. 10 contains a bar graph showing generation of cGMP in human renal proximal tubular cells stimulated by ANP (10⁻¹⁰ M) in absence (Veh) or presence of 1, 5 or 10 μM of compound 19. *P<0.05 vs. Veh.

FIG. 11 contains a bar graph showing generation of cGMP in human visceral adipocytes stimulated by ANP (10⁻¹⁰ M) in absence (Veh) or presence of 1, 5 or 10 μM of compound 19. *P<0.05 vs. Veh.

FIG. 12 contains a bar graph showing generation of cGMP in human subcutaneous adipocytes stimulated by ANP (10⁻¹⁰ M) in absence (Veh) or presence of 1, 5 or 10 μM of compound 19. *P<0.05 vs. Veh.

FIG. 13 contains an image showing cultured human visceral adipocytes stained with Dapi identify the nucleus and LipidSpot to identify lipid droplets. ANP alone reduced lipid droplets. UCPI protein was stained and compound 19 augmented UCPI expression consistent with potentiation of browning by pGC-A.

FIG. 14 ANP alone binding to GC-A. Representative SPR sensorgram for the binding of ANP alone from 0.31 to 5 nM to the extracellular domain of human GC-A resulting in a K_{D} of 715 μM.

FIG. 15 ANP binding to GC-A in the presence of Compound 19. Representative SPR sensorgram for binding of increasing concentrations of ANP from 0.16 to 2.5 nM in the presence 10 μM of Compound 19 to the extracellular domain of human GC-A resulting in a K_{D} of 58 μM.

FIG. 16 Inhibition of Human Cardiomyocyte Hypertrophy with ANP (10⁻¹⁰ M 10⁻⁸ M) and Compound 19. Inhibition of TGF-beta 1 induced human cardiomyocyte hypertrophy by ANP (10⁻¹⁰ M or 10⁻⁸M) alone or in the presence of 1, 5 or 10 μM of compound 19. Vehicle was buffer alone in the absence of TGF-beta 1, ANP or compound 19. *P<0.05 vs. Veh. **P<0.05 vs. TGF-beta 1 alone.

FIG. 17 Plasma cGMP Generation in SHR after IV Bolus of Compound 19. In vivo plasma cGMP levels in SHRs at pre (baseline) and post IV bolus administration of Compound 19.

FIG. 18 Urinary cGMP Excretion in SHR after IV Bolus of Compound 19. In vivo urinary cGMP excretion in SHRs at pre (baseline) and post IV bolus administration of Compound 19.

FIG. 19 Urine Volume Excretion in SHR after IV Bolus of Compound 19. In vivo urinary volume output in SHRs at pre (baseline) and post IV bolus administration of Compound 19.

FIG. 20 Change in MAP after IV Bolus of Compound 19. Change in mean arterial pressure (MAP) in SHRs at 0 min, 15 min, 30 min, 45 min and 60 post IV bolus administration of Compound 19.

FIG. 21 cGMP Generation in HEK GC-A Cell in the presence of Human Plasma & Compound 19. cGMP generation of Compound 19 in human GC-A overexpressing HEK293 cells when incubated with human plasma from normal subjects, hypertensive patients and heart failure patients of which endogenous ANP and BNP levels are present. *P<0.05 vs. Veh within each subject group.

### DETAILED DESCRIPTION

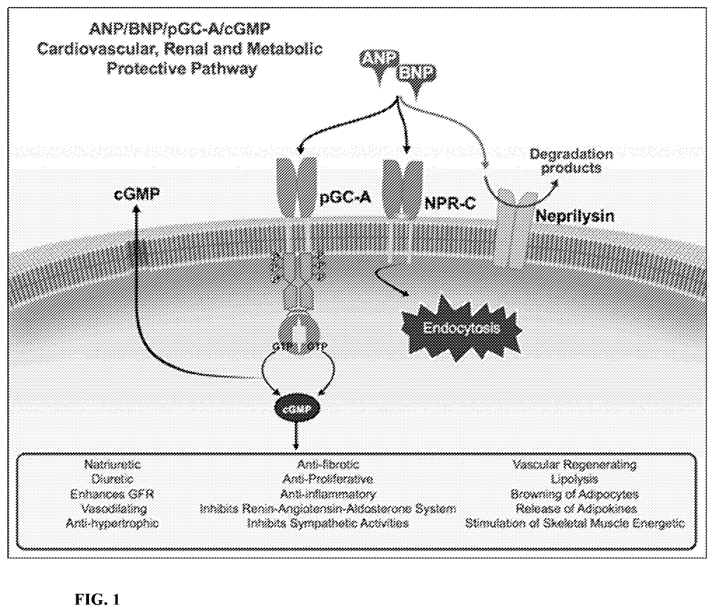

Without being bound by a particular theory, it is believed that the heart is a vital endocrine organ that fine-tunes the body's metabolic homeostasis. Atrial natriuretic peptide (ANP) and B-type natriuretic peptide (BNP) are produced in the heart and released from atrial secretory granules, much like insulin is produced and released from pancreatic secretory granules. The molecular target of these two cardiac hormones is the particulate guanylyl cyclase receptor A (pGC-A) (See FIG. 1) which functions via the second messenger cGMP. Among various physiological functions of pGC-A are regulation of blood pressure (BP), reno-enhancing and renoprotective actions, as well as metabolic actions, including lipolysis with production of non-esterified free fatty acids (NEFA) and glycerol, browning of white adipocytes, stimulation of skeletal muscle energetics, and enhancing release of adipokines such as adiponectin. In one example, in a murine model of obesity, over-expression of the pGC-A activating cardiac hormone BNP protected animals from obesity. In addition, GC-A is highly expressed in the heart, kidney, adrenals, vasculature, and adipocytes. While optimally regulating intravascular volume and blood pressure homeostasis, GC-A activation directly mediates organ protection with anti-apoptotic, anti-fibrotic, anti-hypertrophic, vascular endothelial regenerating, lipolytic, aldosterone suppressing, anti-cancer, and tumor suppressive properties.

Population studies investigating the common genetic variants of the ANP (rs5068) and BNP genes (rs1938845) showed that rs5068 and rs1938845 increase circulating ANP or BNP, respectively. Importantly, the elevation of ANP, through rs5068, was associated with protection from obesity and metabolic syndrome, decreased waist circumference, higher HDL levels with reduced BP, and risk for hypertension. While rs5068 is common, only 10% of the population carry this ANP genetic variant, and exhibit the protective phenotype. Hence, approximately 90% of the population has a relative higher risk for metabolic syndrome and hypertension based upon this ANP genetic variation. It was also shown that metabolic protective actions of rs5068 are present in African Americans, which highlights the multiethnic metabolic protection of pGC-A. Furthermore, the BNP gene variant, rs1938845 was found to be associated with reduced risk for type II diabetes mellitus as well as prolonged survival of the diabetes patients. Importantly, in patients with chronic heart failure, twice daily subcutaneously (SQ) administered BNP and subsequently GC-A activation reversed cardiac hypertrophy and improved myocardial function and notably, improved patient symptoms.

Without being bound by a theory, it is believed that the compounds described herein increase pGC-A responsiveness to the endogenous ligands (ANP and BNP), even at reduced levels, by enhancing the pGC-A function in a positive allosteric manner. The compounds within the present claims also exhibited good ADME (Absorption, Distribution, Metabolism, and Excretion) properties including solubility, microsomal stability and plasma stability.

Therapeutic Compounds

In a general aspect, the present disclosure provides a compound of Formula (I):

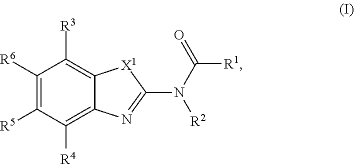

or a pharmaceutically acceptable salt thereof, wherein:

X¹ is selected from S, O, and NR²;

R¹ is selected from any one of the following groups:

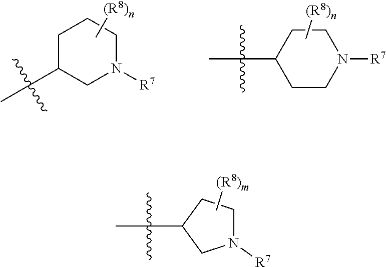

R² is selected from H and C₁₋₃ alkyl;

R³ and R⁵ are each independently selected from H, halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} and S(O)₂NR^{c1}R^{d1};

R⁴ is halo;

R⁶ is selected from H and halo;

each R⁷ is independently selected from H, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl, C₂₋₆ alkenyl, and C₂₋₆ alkynyl are each optionally substituted with 1, 2 or 3 substituents independently selected from Cy¹, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};

each Cy¹ is independently selected from C₆₋₁₀ aryl and C₃₋₁₀ cycloalkyl, each of which is optionally substituted with 1, 2, 3, 4, or 5 substituents independently selected from R^{Cy1};

each R^{Cy1} is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};

each R⁸ is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{c1})NR^{c1}R^{d1}, NR^{c1}C(═NR^{c1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};

each n is independently 0, 1, 2, 3, 4, 5, 6, 7, or 8;

M is 0, 1,2, 3, 4, 5, or 6;

each R^{a1} and R^{b1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene are optionally substituted with 1, 2, 3, 4, or 5 substituents independently selected from R^{g};

each R^{c1} and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, C₃₋₁₀ cycloalkyl-C₁₋₄ alkylene, C(O)R^{b2}, C(O)NR^{c2}R^{d2}, C(O)OR^{a2}, NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene is optionally substituted with 1, 2, 3, 4, or 5 substituents independently selected from R^{g};

each R^{a2}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, C₃₋₁₀ cycloalkyl-C₁₋₄ alkylene, and R^{g}, wherein said C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene is optionally substituted with 1, 2, 3, 4, or 5 substituents independently selected from R^{g};

or any R^{c1} and R^{d1} together with the N atom to which they are attached form a 4-, 5-, 6-, or 7-membered heterocycloalkyl group optionally substituted with 1, 2, or 3 substituents independently selected from R^{g};

or any R^{c2} and R^{d2} together with the N atom to which they are attached form a 4-, 5-, 6-, or 7-membered heterocycloalkyl group optionally substituted with 1, 2, or 3 substituents independently selected from R^{g};

each R^{c1} is independently selected from H, C₁₋₄ alkyl, C₁₋₄ alkoxy, OH, and CN; and

each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, (C₃₋₁₀ cycloalkyl)amino, di(C₃₋₁₀cycloalkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

In some embodiments, R³ and R⁵ are each independently selected from H, halo, CN, NO₂, C₁₋₆ alkyl, C₁₋₆ haloalkyl, OR^{a1}, wherein said C₁₋₆ alkyl is optionally substituted with CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} or S(O)₂NR^{c1}R^{d1}.

In some embodiments, R³ and R⁵ are each independently selected from H, halo, C₁₋₆ alkyl, C₁₋₆ haloalkyl, C₁₋₆ alkoxy, and C₁₋₆ haloalkoxy.

In some embodiments, R³ is H and R⁵ is selected from halo, C₁₋₆ alkyl, C₁₋₆ haloalkyl, C₁₋₆ alkoxy, and C₁₋₆ haloalkoxy. In some embodiments, R³ is selected from halo, C₁₋₆ alkyl, C₁₋₆ haloalkyl, C₁₋₆ alkoxy, and C₁₋₆ haloalkoxy, and R⁵ is H.

In some embodiments, R³ and R⁵ are each H.

In some embodiments, R⁴ is selected from Cl and F. In some aspects of these embodiments, R⁴ is Cl. In other aspects of these embodiments, R⁴ is F.

In some embodiments, R⁶ is H.

In some embodiments, R⁶ is halo. In some aspects of these embodiments, R⁶ is Cl. In other aspects of these embodiments, R⁶ is F.

In some embodiments, X¹ is S.

In some embodiments, X¹ is NH.

In some embodiments, X¹ is O.

In some embodiments, the compound of Formula (I) has formula:

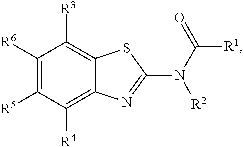

or a pharmaceutically acceptable salt thereof.

In some embodiments, the compound of Formula (I) has formula:

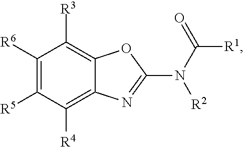

or a pharmaceutically acceptable salt thereof.

In some embodiments, the compound of Formula (I) has formula:

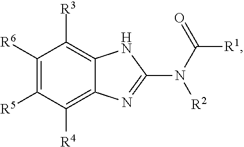

or a pharmaceutically acceptable salt thereof.

In some embodiments, the compound of Formula (I) has formula:

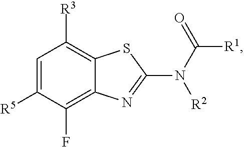

or a pharmaceutically acceptable salt thereof.

In some embodiments, the compound of Formula (I) has formula:

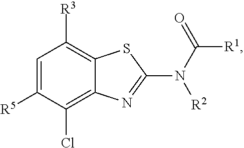

or a pharmaceutically acceptable salt thereof.

In some embodiments, the compound of Formula (I) has formula:

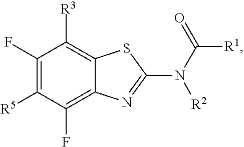

or a pharmaceutically acceptable salt thereof.

In some embodiments, R⁴ is Cl and R⁶ is F. In other embodiments, R⁴ is F and R⁶ is Cl. In yet other embodiments, R⁴ is Cl and R⁶ is Cl.

In some embodiments, R² is H.

In some embodiments, R² is C₁₋₃ alkyl (e.g., methyl, ethyl, propyl, or isopropyl).

In some embodiments, each le is independently selected from halo, CN, NO₂, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl is optionally substituted with halo, CN, NO₂, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} or S(O)₂NR^{c1}R^{d1}.

In some embodiments, each le is independently selected from halo, C₁₋₆ alkyl, C₁₋₆ haloalkyl, C₁₋₆ alkoxy, and C₁₋₆ haloalkoxy.

In some embodiments, each n is independently 1 or 2. In some aspects of these embodiments, n is 1. In other aspects of these embodiments, n is 2. In some embodiments, n is 0.

In some embodiments, m is 1 or 2. In some aspects of these embodiments, m is 1. In other aspects of these embodiments, m is 2. In some embodiments, m is 0.

In some embodiments, each n is 0 and m is 0.

In some embodiments:

R³ and R⁵ are each independently selected from H, halo, CN, NO₂, C₁₋₆ alkyl, C₁₋₆ haloalkyl, OR^{a1}, wherein said C₁₋₆ alkyl is optionally substituted with CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} or S(O)₂NR^{c1}R^{d1};

each R⁸ is independently selected from halo, CN, NO₂, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl is optionally substituted with halo, CN, NO₂, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} or S(O)₂NR^{c1}R^{d1};

each n is independently 1 or 2; and

m is 1 or 2.

In some embodiments:

R³ and R⁵ are each independently selected from H, halo, C₁₋₆ alkyl, C₁₋₆ haloalkyl, C₁₋₆ alkoxy, and C₁₋₆ haloalkoxy;

each R⁸ is independently selected from halo, C₁₋₆ alkyl, C₁₋₆ haloalkyl, C₁₋₆ alkoxy, and C₁₋₆ haloalkoxy;

R² is H;

each n is independently 1 or 2; and

m is 1 or 2.

In some embodiments:

R³ and R⁵ are each H;

R² is H;

each n is 0;

and m is 0.

In some embodiments, R¹ is:

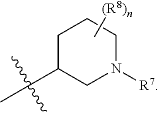

In some embodiments, R¹ is:

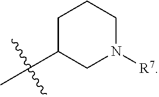

In some embodiments, R¹ is:

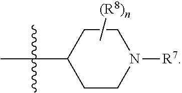

In some embodiments, R¹ is:

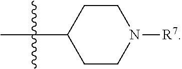

In some embodiments, R¹ is:

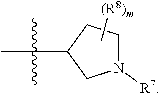

In some embodiments, R¹ is:

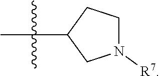

In some embodiments, the compound of Formula (I) has formula:

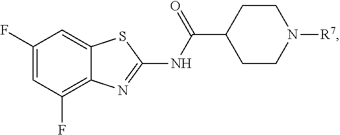

or a pharmaceutically acceptable salt thereof.

In some embodiments, the compound of Formula (I) has formula:

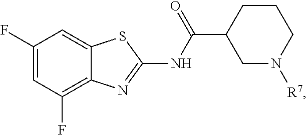

or a pharmaceutically acceptable salt thereof.

In some embodiments, the compound of Formula (I) has formula:

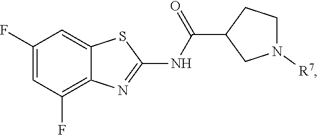

or a pharmaceutically acceptable salt thereof.

In some embodiments, R⁷ is selected from H, C₁₋₆ alkyl, C₁₋₆ haloalkyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with Cy¹, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1}.

In some embodiments, R⁷ is selected from H, C₁₋₆ alkyl, C₁₋₆ haloalkyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl is optionally substituted with C₆₋₁₀ aryl, OR^{a1}, OC(O)R^{b1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂nR^{c1}R^{d1}, S(O)₂R^{b1} and S(O)₂NR^{c1}R^{d1}.

In some embodiments, R⁷ is selected from H, C₁₋₆ alkyl, C(O)R^{b1}, C(O)OR^{a1}, and S(O)₂R^{b1}, wherein said C₁₋₆ alkyl is optionally substituted with C₆₋₁₀ aryl or NR^{c1}R^{d1}.

In some embodiments, R⁷ is H.

In some embodiments, R⁷ is C₁₋₆ alkyl.

In some embodiments, R⁷ is C(O)R^{b1}. In some embodiments, R^{b1} is C₁₋₆ alkyl optionally substituted with amino. In some embodiments, R^{b1} is C₁₋₆ alkyl optionally substituted with C₁₋₆ alkylamino. In some embodiments, R^{b1} is C₁₋₆ alkyl optionally substituted with di(C₁₋₆ alkyl)amino. In some embodiments, R^{b1} is C₁₋₆ alkyl optionally substituted with (C₃₋₁₀ cycloalkyl)amino. In some embodiments, R^{b1} is C₁₋₆ alkyl optionally substituted with di(C₃₋₁₀ cycloalkyl)amino.

In some embodiments, R⁷ is C(O)OR^{a1}.

In some embodiments, R⁷ is S(O)₂R^{b1}.

In some embodiments, R⁷ is C₁₋₆ alkyl substituted with OR^{a1}, OC(O)R^{b1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, or S(O)₂NR^{c1}R^{d1}.

In some embodiments, R⁷ is C₁₋₆ alkyl substituted with OR^{a1}, C(O)OR^{a1}, NR^{c1}R^{d1}, NR^{c1}S(O)₂R^{b1}, S(O)₂R^{b1}, or S(O)₂NR^{c1}R^{d1}.

In some embodiments, R⁷ is C₁₋₆ alkyl substituted with OR^{a1}, C(O)OR^{a1}, or NR^{c1}R^{d1}.

In some embodiments, R⁷ is C₁₋₆ alkyl substituted with NR^{c1}R^{d1}. In some embodiments, R^{c1} and R^{d1} are each independently selected from H, C₁₋₆ alkyl, and C₃₋₁₀ cycloalkyl.

In some embodiments, R⁷ is C₁₋₆ alkyl substituted with Cy¹.

In some embodiments, Cy¹ is C₆₋₁₀ aryl, optionally substituted with 1, 2, or 3 R^{Cy1}.

In some embodiments, Cy¹ is C₃₋₁₀ cycloalkyl, optionally substituted with 1, 2, or 3 R^{Cy1}.

In some embodiments, R⁷ is C₁₋₆ alkyl substituted with C₆₋₁₀ aryl, which is optionally substituted with 1, 2, or 3 R. In some embodiments, each R^{Cy1} is independently selected from halo, CN, NO₂, C₁₋₆ alkyl, C₁₋₆ haloalkyl, OR^{a1}, C(O)R^{b1}, C(O)NR^{c1}R^{d1}, C(O)OR^{a1}, and NR^{c1}R^{d1}; wherein said C₁₋₆ alkyl is optionally substituted with 1, 2 or 3 substituents independently selected from halo, CN, NO₂, OR^{a1}, C(O)R^{b1}, C(O)NR^{c1}R^{d1}, C(O)OR^{a1}, and NR^{c1}R^{d1}.

In some embodiments, each R^{Cy1} is independently selected from halo, OH, amino, C₁₋₆ alkyl, C₁₋₆ haloalkyl, C₁₋₆ alkoxy, and C₁₋₆ haloalkoxy.

In some embodiments, each R^{a1} and R^{b1} is independently selected from C₁₋₆ alkyl, C₆₋₁₀ aryl, C₃₋₁₀ cycloalkyl, and C₆₋₁₀ aryl-C₁₋₄ alkylene, wherein said C₁₋₆ alkyl, C₆₋₁₀ aryl, C₃₋₁₀ cycloalkyl, and C₆₋₁₀ aryl-C₁₋₄ alkylene are each optionally substituted with 1, 2, or 3 substituents independently selected from R^{g}.

In some embodiments, R^{b1} is selected from C₁₋₆ alkyl, C₆₋₁₀ aryl, C₃₋₁₀ cycloalkyl, and C₆₋₁₀ aryl-C₁₋₄ alkylene, each of which is optionally substituted with R^{g}. In some embodiments, R^{b1} is C₁₋₆ alkyl. In some embodiments, R^{b1} is C₆₋₁₀ aryl. In some embodiments, R^{b1} is C₃₋₁₀ cycloalkyl. In some embodiments, R^{b1} is C₆₋₁₀ aryl-C₁₋₄ alkylene.

In some embodiments, each R^{g} is independently selected from OH, NO₂, CN, halo, C₁₋₆ alkyl, C₁₋₄ haloalkyl, C₁₋₆ alkoxy, C₁₋₆ haloalkoxy, amino, C₁₋₆ alkylamino, and di(C₁₋₆ alkyl)amino.

In some embodiments, the compound of Formula (I) is selected from any one of the following compounds:

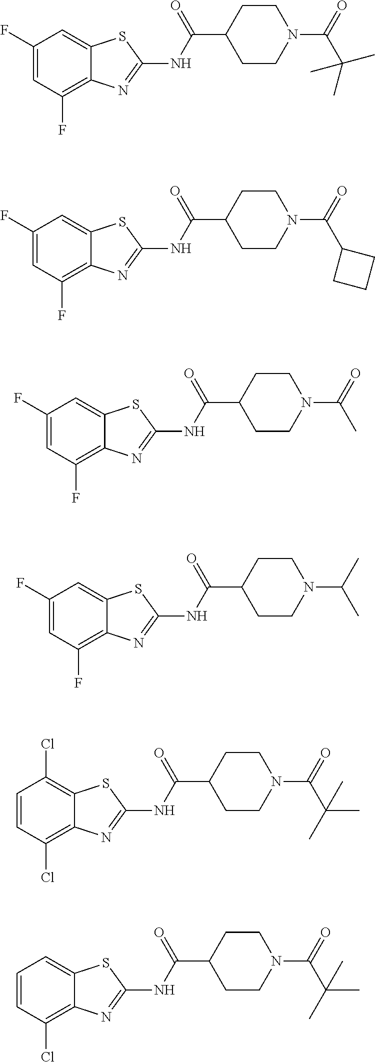 [SMILES: CC(C)(C)C(=O)N1CCC(C(=O)Nc2nc3c(F)cc(F)cc3s2)CC1 ; O=C(Nc1nc2c(F)cc(F)cc2s1)C1CCN(C(=O)C2CCC2)CC1 ; CC(=O)N1CCC(C(=O)Nc2nc3c(F)cc(F)cc3s2)CC1 ; CC(C)N1CCC(C(=O)Nc2nc3c(F)cc(F)cc3s2)CC1 ; CC(C)(C)C(=O)N1CCC(C(=O)Nc2nc3c(Cl)ccc(Cl)c3s2)CC1 ; CC(C)(C)C(=O)N1CCC(C(=O)Nc2nc3c(Cl)cccc3s2)CC1]

or a pharmaceutically acceptable salt thereof.

In some embodiments, the compound of Formula (I) is selected from any one of the following compounds:

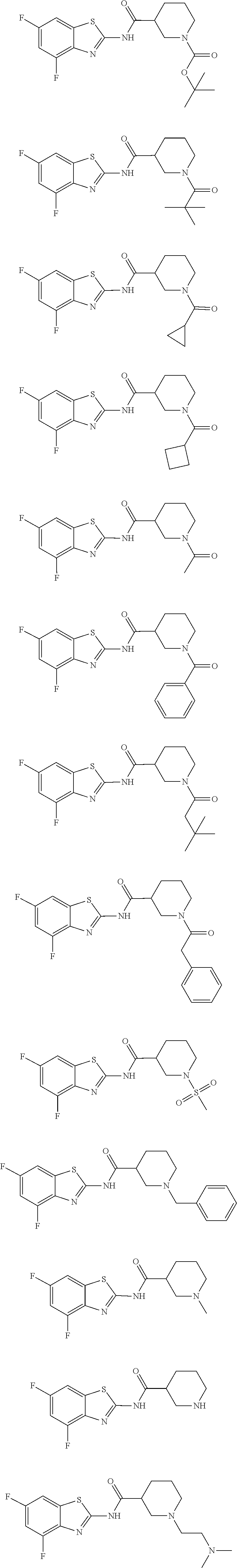

or a pharmaceutically acceptable salt thereof.

In some embodiments, the compound of Formula (I) is selected from any one of the following compounds:

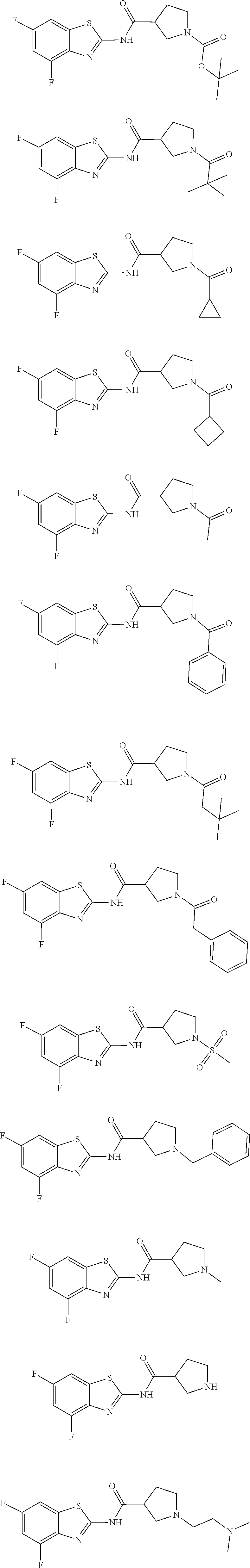

or a pharmaceutically acceptable salt thereof.

Pharmaceutically Acceptable Salts

In some embodiments, a salt of a compound of Formula (I) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of the present disclosure include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like. In some embodiments, the compounds of Formula (I), or pharmaceutically acceptable salts thereof, are substantially isolated.

Methods of Making Therapeutic Compounds

Compounds of Formula (I), including salts thereof, can be prepared using known organic synthesis techniques and can be synthesized according to any of numerous possible synthetic routes. A person skilled in the art knows how to select and implement appropriate synthetic protocols, and appreciates that the processes described are not the exclusive means by which compounds provided herein may be synthesized, and that a broad repertoire of synthetic organic reactions is available to be potentially employed in synthesizing compounds provided herein.

Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

Methods of Using Therapeutic Compounds

The present disclosure provides, at least in part, that the pGC-A/cGMP pathway is a valuable molecular target for metabolic, cardiovascular (CV), renal, and anticancer therapeutics. As discussed above, the elevation of pGC-A's endogenous ligand ANP levels is associated with protection from obesity and metabolic syndrome, reduced blood pressure, decreased risk for hypertension as well as reduced incidence of myocardial infarction. Similarly, the elevation of levels of endogenous ligand BNP is associated with reduced risk for type II diabetes mellitus.

Accordingly, in a general aspect, the present disclosure provides a method of modulating particulate guanylyl cyclase receptor A (pGC-A) in a cell, the method comprising contacting the cell with an effective amount of the compound of Formula (I), or a pharmaceutically acceptable salt thereof. In some embodiments, the cell is contacted in vitro, in vivo, or ex vivo.

The present disclosure also provides a method of modulating particulate guanylyl cyclase receptor A (pGC-A) in a subject, the method comprising administering to the subject in need thereof an effective amount of the compound of Formula (I), or a pharmaceutically acceptable salt thereof, or the pharmaceutical composition comprising same.

In some embodiments of the methods of the present disclosure, modulating of the particulate guanylyl cyclase receptor A (pGC-A) comprises positive allosteric enhancement of activity of the particulate guanylyl cyclase receptor A (pGC-A) (e.g., the modulating comprises increased production cGMP in a cell (e.g., in a cell of the subject)). In some embodiments, the cell is a renal cell or a heart muscle cell.

The present disclosure also provides a method of treating or preventing a disease or condition responsive to modulation of a particulate guanylyl cyclase receptor A (pGC-A) in a subject, the method comprising administering to the subject in need thereof a therapeutically effective amount of the compound of Formula (I), or a pharmaceutically acceptable salt thereof, or the pharmaceutical composition comprising same.

The present disclosure also provides a compound of Formula (I), or a pharmaceutically acceptable salt thereof, or the pharmaceutical composition comprising same, for use in a manufacture of a medicament for the treatment or prevention of a disease or condition responsive to modulation of a particulate guanylyl cyclase receptor A (pGC-A) in a subject.

The present disclosure also provides a compound of Formula (I), or a pharmaceutically acceptable salt thereof, or the pharmaceutical composition comprising same, for use the treatment or prevention of a disease or condition responsive to modulation of a particulate guanylyl cyclase receptor A (pGC-A) in a subject.

In some embodiments, the disease or condition responsive to modulation of a particulate guanylyl cyclase receptor A (pGC-A) is a metabolic disease or disorder. In some embodiments, the metabolic disorder is congenital. Suitable examples of such disorders include Fabry disease, phenylketonuria, Prader-Willi syndrome, galactosemia, Tay-Sachs's disease, porphyria, Pompe disease, Neimann-Pick disease, Morquio's syndrome, Morteaus-lamy syndrome, Hunter syndrome, Lesh-Nyhan syndrome, Hurler syndrome, homocystinuria, Hartnup disease, and Gaucher's disease. In some embodiments, the metabolic disorder is acquired. Suitable examples of such disorders include diabetes (e.g., type 1 diabetes, diabetes insipidus, or type II diabetes mellitus), obesity, metabolic syndrome, dyslipidemia, hipolipidemia (hyperlipoproteinemia), hyperthyroidism, hypoparathyroidism, hypothyroidism, Cushing's syndrome, hyperuricemia, hemochromatosis, and hyperparathyroidism. Other examples of metabolic disorders include glucose intolerance, insulin resistance, fibrinolysis disorder, endothelial dysfunction, atherosclerosis, impaired fasting glycemia, hyperinsulinemia, galactosemia, mucopolysaccaridose, tyrosinemia, methylmalonic aciduria, acidemia (e.g., propionic acidemia, isovaleric acidemia), and hyperammonemia. In some embodiments, the metabolic disease is selected from obesity, hypertriglyceridemia, metabolic syndrome, insulin resistance, hyperinsulinemia, diabetes, and acidemia.

In some embodiments, the disease or condition responsive to modulation of a particulate guanylyl cyclase receptor A (pGC-A) is a cardiovascular disease. Suitable examples of cardiovascular disorders include high blood pressure, myocardial infarction, abnormal heart rhythms (e.g., arrhythmia), aorta disease, Marfan syndrome, congenital heart disease, coronary artery disease (e.g., narrowing of the arteries), deep vein thrombosis, pulmonary embolism, heart attack, heart failure, heart muscle disease (e.g., cardiomyopathy), heart valve disease, pericardial disease, peripheral vascular disease, rheumatic heart disease, stroke, vascular disease (e.g., blood vessel disease), cardiomyopathies, hypertension, aortic stenosis, mitral valve insufficiency, mitral valve prolapse, pericarditis, rheumatic heart disease, and cardiorenal syndrome. In some embodiments, the cardiovascular disease is selected from heart failure, cardiomyopathy, hypertension, high blood pressure, and myocardial infarction.

In some embodiments, the disease or condition responsive to modulation of a particulate guanylyl cyclase receptor A (pGC-A) is kidney disease. Suitable examples of renal diseases include nephropathy, acute kidney injury, kidney failure, acute renal failure, kidney stones, glomerulonephritis, polycystic kidney disease, urinary tract infections, kidney infection (pyelonephritis), simple kidney cysts, diabetic kidney disease, nephropathy, lupus nephritis, Henoch-Schonlein purpura, goodpasture syndrome, ectopic kidney, amyloidosis, acquired cystic kidney disease, glomerular disease, kidney dysplasia, medullary sponge kidney, nephrotic syndrome, kidney damage, renal artery stenosis, renal tubular acidosis, and solitary kidney. In some embodiments, the kidney disease is selected from nephropathy, acute renal failure, chronic kidney disease, cardiorenal syndrome and diabetic kidney disease.

In some embodiments, the disease or condition responsive to modulation of a particulate guanylyl cyclase receptor A (pGC-A) is cancer. Suitable example of cancer include bladder cancer, brain cancer, breast cancer, colorectal cancer (e.g., colon cancer), rectal cancer, cervical cancer, gastrointestinal cancer, genitourinary cancer, head and neck cancer, lung cancer, oral cancer, ovarian cancer, pancreatic cancer (e.g., pancreatic neuroendocrine tumor), prostate cancer, endometrial cancer, renal cancer (kidney cancer) (e.g., advanced kidney cancer), skin cancer, liver cancer, thyroid cancer, leukemia, and testicular cancer.

Pharmaceutical Compositions and Formulations

The present application also provides pharmaceutical compositions comprising an effective amount of a compound of Formula (I), or a pharmaceutically acceptable salt thereof, and a pharmaceutically acceptable carrier. The pharmaceutical composition may also comprise any one of the additional therapeutic agents described herein, or a pharmaceutically acceptable salt thereof. In certain embodiments, the application also provides pharmaceutical compositions and dosage forms comprising any one the additional therapeutic agents described herein, or a pharmaceutically acceptable salt thereof, and a pharmaceutically acceptable carrier. The carrier(s) and excipient(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, a compound of Formula (I) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a compound of Formula (I) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent in a suitable amount or dosage.

Definitions

At various places in the present specification, substituents of compounds of the present application are disclosed in groups or in ranges. It is specifically intended that various embodiments of the present application include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, the term “about” means “approximately” (e.g., plus or minus approximately 10% of the indicated value).

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified.

As used herein, the term “tautomer” refers to compounds which are capable of existing in a state of equilibrium between two isomeric forms. Such compounds may differ in the bond connecting two atoms or groups and the position of these atoms or groups in the compound.

As used herein, the term “isomer” refers to structural, geometric and stereo isomers.

Throughout the definitions, the term “Cₙ₋ₘ” indicates a range which includes the endpoints, wherein n and m are integers and indicate the number of carbon atoms. Examples include C₁₋₄, C₁₋₆, and the like.

As used herein, the phrase “optionally substituted” means unsubstituted or substituted. As used herein, the term “substituted” means that a hydrogen atom is removed and replaced by a substituent. It is to be understood that substitution at a given atom is limited by valency.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “C.haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (e.g., n-propoxycarbonyl and isopropoxycarbonyl), butoxycarbonyl (e.g., n-butoxycarbonyl and tent-butoxycarbonyl), and the like.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (e.g., n-propylcarbonyl and isopropylcarbonyl), butylcarbonyl (e.g., n-butylcarbonyl and tent-butylcarbonyl), and the like.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “C-m alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “carbamyl” to a group of formula —C(O)NH₂.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ-alkyl)carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “carbonyl”, employed alone or in combination with other terms, refers to a —C(═O)— group, which may also be written as C(O).

As used herein, the term “carboxy” refers to a —C(O)OH group.

As used herein, the term “cyano-C₁₋₃ alkyl” refers to a group of formula —(C₁₋₃ alkylene)-CN.

As used herein, the term “HO—C₁₋₃ alkyl” refers to a group of formula —(C₁₋₃ alkylene)-OH.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system, an in vivo system, or an ex vivo system. For example, “contacting” the particulate guanylyl cyclase receptor A with a compound of the invention includes the administration of a compound of the present invention to an individual or patient, such as a human, having particulate guanylyl cyclase receptor A, as well as, for example, introducing a compound of the invention into a sample containing a cellular or purified preparation containing the particulate guanylyl cyclase receptor A.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

Assay: Generally, the assay monitors the production of cGMP, the second messenger generated by pGC-A, by Time-Resolved Florescence (HTRF) in HEK293 cells overexpressing the pGC-A. pGC-A suspension cells were stimulated in the presence of the test compound and an EC₂₀ concentration of ANP. The quantity of cGMP was detected by competitive immunoassay using Eu³⁺ cryptate-labeled anti-cGMP and d2-labeled cGMP and normalized to maximal amount produced by an EC₈₀ concentration of ANP. Compound EC₅₀ values were determined in the primary assay, in the presence or absence of ANP, to determine mode of action as positive modulators and tested for selectivity in the same assay platform but in HEK cells overexpressing the particulate guanylyl cyclase B receptor (pGC-B), of which CNP is the endogenous ligand.

Assay details: 20 nL of 10 μM test compound in DMSO was added to columns 5-48 of 1536 well white high base screening plates (Corning, New York, N.Y.) cells using 550 ECHO acoustic dispenser (Labcyte, San Jose, Calif.). Alpha-atrial natriuretic peptide (ANP) (Phoenix Pharmaceutics) was prepared as stock aliquots at 5 μM in PBS with 0.1% BSA. An approximate EC₃₀ concentration of ANP (9 pM) in assay buffer (HBSS containing 5 mM HEPES and 0.05% BSA) was added to columns 3-48 at a volume of 1 μL. Assay buffer only was added to column 1 and assay buffer containing a saturating concentration of ANP (5 nM) was added to column 2. HEK293 cells overexpressing GC-A were resuspended in assay media (OptimMem media containing 2% Heat-inactivated Fetal bovine serum and L-glutamine) at a density of 6×10⁵ cells/mL and 2 μL were plated in screening plates (1200 cells/well) in suspension using a Bioraptr 2. Plates were spun at 1000 rpm for 1 min and incubated for 30 min at room temperature. 1.5 μL d2-labled cGMP followed by 1.5 uL Eu³⁺ cryptate-labeled anti-cGMP cGMP detection kit (CiBio; #62GM2PEC) prepared according to manufacturer's protocol were added to all wells using a Bioraptr 2 and TR-FRET signal was detected on an EnVision detector (PerkinElmer). Wells treated with 0.3% DMSO only serve as blank controls (column 1); wells treated with 0.3% DMSO and 5 nM ANP (columns 2) serve as positive controls and wells treated with 0.3% DMSO and 9 pM ANP (columns 3-4) serve as negative controls. DMSO did not exceed 0.3% in all wells.

### Example 1

Initial screen led to identification of a compound A:

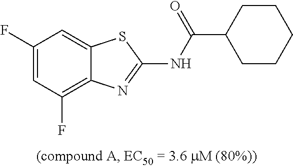 [SMILES: O=C(Nc1nc2c(F)cc(F)cc2s1)C1CCCCC1]

In contrast, the compound 1 of the present disclosure:

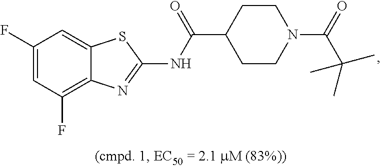 [SMILES: CC(C)(C)C(=O)N1CCC(C(=O)Nc2nc3c(F)cc(F)cc3s2)CC1]

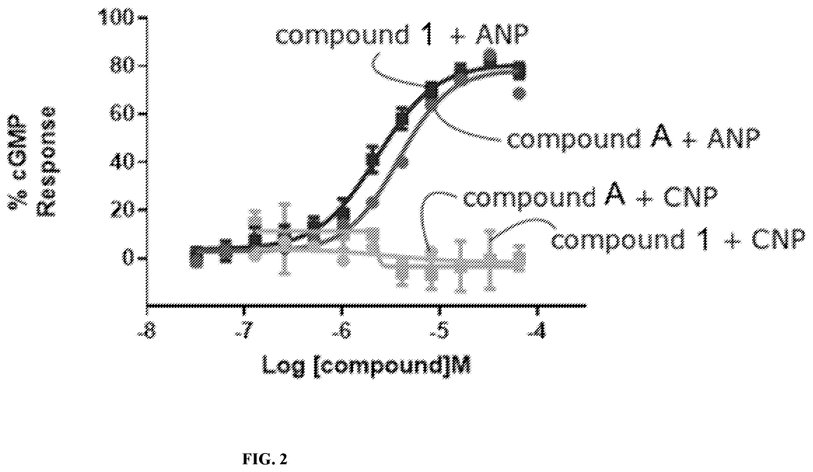

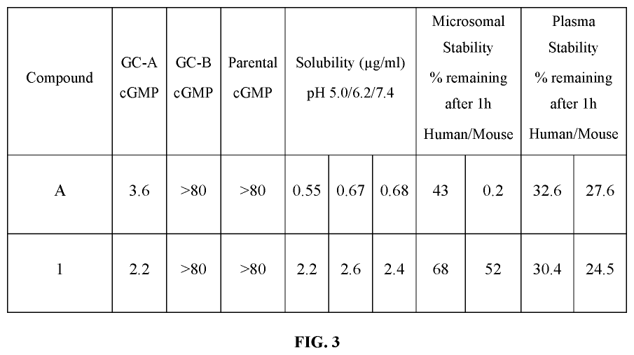

shows improved potency, solubility, and rodent microsomal stability compared to compound A (See FIG. 2-3).

### Example 2

Test results (EC₅₀, max efficacy) for the piperidine-4-carboxamide exemplified compounds are shown in Table 1.

TABLE 1

| Cmpd. | | EC₅₀ | Max |
|---|---|---|---|
| No. | Structure | (μM) | efficacy (%) |
| 1 | 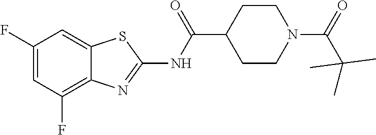 [SMILES: CC(C)(C)C(=O)N1CCC(C(=O)Nc2nc3c(F)cc(F)cc3s2)CC1] | 2.18, 1.38, 1.57 | 83, 76, 77 |
| 2 | 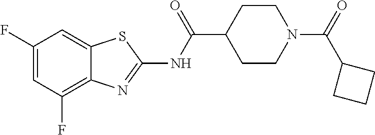 [SMILES: O=C(Nc1nc2c(F)cc(F)cc2s1)C1CCN(C(=O)C2CCC2)CC1] | 1.72 | 79 |
| 3 | 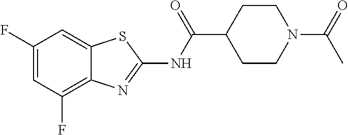 [SMILES: CC(=O)N1CCC(C(=O)Nc2nc3c(F)cc(F)cc3s2)CC1] | 1.15 | 74 |
| 4 | 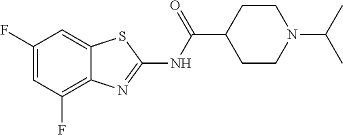 [SMILES: CC(C)N1CCC(C(=O)Nc2nc3c(F)cc(F)cc3s2)CC1] | 0.84 | 80 |
| 5 | 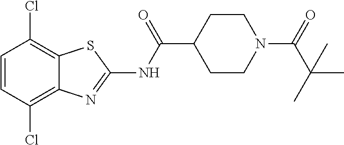 [SMILES: CC(C)(C)C(=O)N1CCC(C(=O)Nc2nc3c(Cl)ccc(Cl)c3s2)CC1] | >66 | n/a |
| 6 | 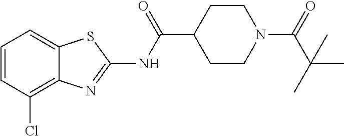 [SMILES: CC(C)(C)C(=O)N1CCC(C(=O)Nc2nc3c(Cl)cccc3s2)CC1] | 2.00 | 51 |

### Example 3

Test results (EC₅₀, max efficacy) for the piperidine-3-carboxamide exemplified compounds are shown in Table 2.

TABLE 2

| Cmpd. | | EC₅₀ | Max efficacy |
|---|---|---|---|
| No. | Structure | (μM) | (%) |
| 7 | 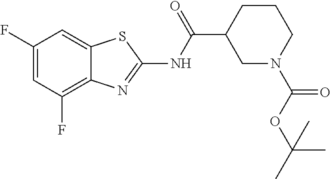 [SMILES: CC(C)(C)OC(=O)N1CCCC(C(=O)Nc2nc3c(F)cc(F)cc3s2)C1] | 8.26 | 64 |
| 8 | 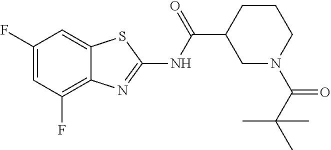 [SMILES: CC(C)(C)C(=O)N1CCCC(C(=O)Nc2nc3c(F)cc(F)cc3s2)C1] | 5.07 | 73 |
| 9 | 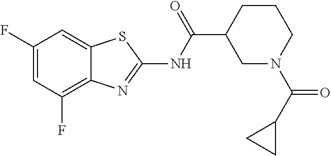 [SMILES: O=C(Nc1nc2c(F)cc(F)cc2s1)C1CCCN(C(=O)C2CC2)C1] | 1.97 | 82 |
| 10 | 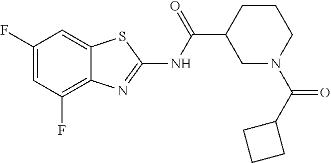 [SMILES: O=C(Nc1nc2c(F)cc(F)cc2s1)C1CCCN(C(=O)C2CCC2)C1] | 1.81 | 77 |
| 11 | 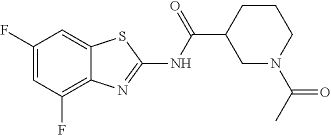 [SMILES: CC(=O)N1CCCC(C(=O)Nc2nc3c(F)cc(F)cc3s2)C1] | 1.65 | 79 |
| 12 | 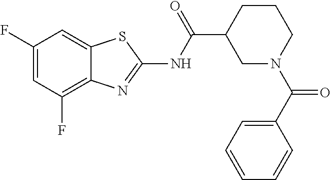 [SMILES: O=C(Nc1nc2c(F)cc(F)cc2s1)C1CCCN(C(=O)c2ccccc2)C1] | 3.36 | 64 |
| 13 | 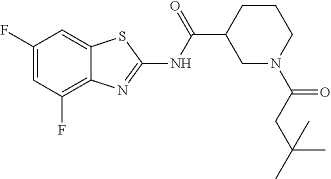 [SMILES: CC(C)(C)CC(=O)N1CCCC(C(=O)Nc2nc3c(F)cc(F)cc3s2)C1] | 3.57 | 74 |
| 14 | 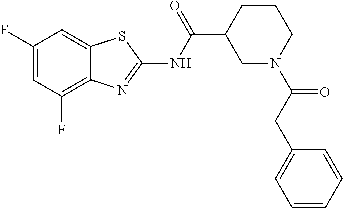 [SMILES: O=C(Nc1nc2c(F)cc(F)cc2s1)C1CCCN(C(=O)Cc2ccccc2)C1] | 2.02 | 71 |
| 15 | 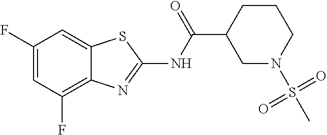 [SMILES: CS(=O)(=O)N1CCCC(C(=O)Nc2nc3c(F)cc(F)cc3s2)C1] | 1.75 | 77 |
| 16 | 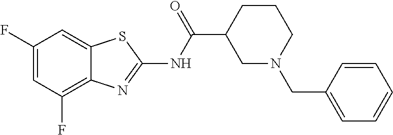 [SMILES: O=C(Nc1nc2c(F)cc(F)cc2s1)C1CCCN(Cc2ccccc2)C1] | 2.41 | 66 |
| 17 | 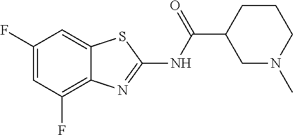 [SMILES: CN1CCCC(C(=O)Nc2nc3c(F)cc(F)cc3s2)C1] | 1.23 | 81 |
| 18 | 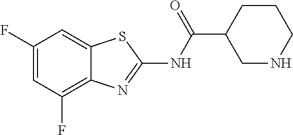 [SMILES: O=C(Nc1nc2c(F)cc(F)cc2s1)C1CCCNC1] | 1.05 | 82 |
| 19 | 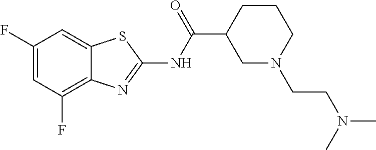 [SMILES: CN(C)CCN1CCCC(C(=O)Nc2nc3c(F)cc(F)cc3s2)C1] | 0.39 | 82 |

### Example 4

Test results (EC₅₀, max efficacy) for the pyrrolidine-3-carboxamide exemplified compounds are shown in Table 3.

TABLE 3

| Cmpd. | | EC₅₀ | Max efficacy |
|---|---|---|---|
| No. | Structure | (μM) | (%) |
| 20 | 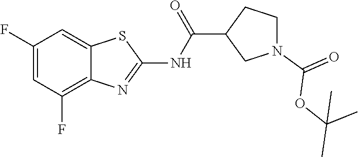 [SMILES: CC(C)(C)OC(=O)N1CCC(C(=O)Nc2nc3c(F)cc(F)cc3s2)C1] | 4.38 | 74 |
| 21 | 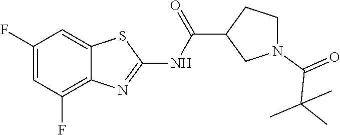 [SMILES: CC(C)(C)C(=O)N1CCC(C(=O)Nc2nc3c(F)cc(F)cc3s2)C1] | 2.09 | 84 |
| 22 | 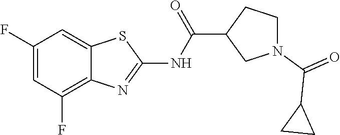 [SMILES: O=C(Nc1nc2c(F)cc(F)cc2s1)C1CCN(C(=O)C2CC2)C1] | 6.25 | 82 |
| 23 | 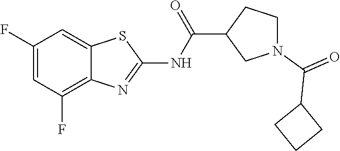 [SMILES: O=C(Nc1nc2c(F)cc(F)cc2s1)C1CCN(C(=O)C2CCC2)C1] | 6.06 | 82 |
| 24 | 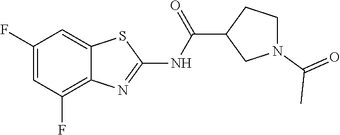 [SMILES: CC(=O)N1CCC(C(=O)Nc2nc3c(F)cc(F)cc3s2)C1] | 0.56 | 84 |
| 25 | 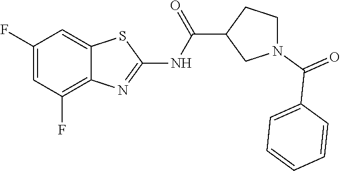 [SMILES: O=C(Nc1nc2c(F)cc(F)cc2s1)C1CCN(C(=O)c2ccccc2)C1] | 6.86 | 84 |
| 26 | 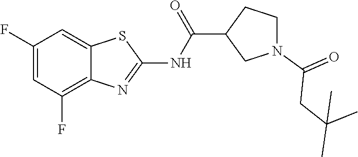 [SMILES: CC(C)(C)CC(=O)N1CCC(C(=O)Nc2nc3c(F)cc(F)cc3s2)C1] | 3.23 | 81 |
| 27 | 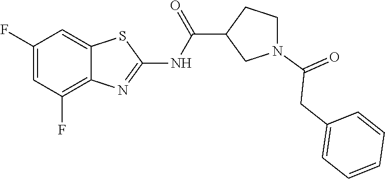 [SMILES: O=C(Nc1nc2c(F)cc(F)cc2s1)C1CCN(C(=O)Cc2ccccc2)C1] | 2.69 | 66 |
| 28 | 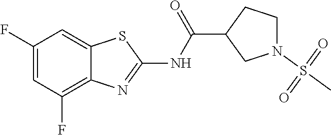 [SMILES: CS(=O)(=O)N1CCC(C(=O)Nc2nc3c(F)cc(F)cc3s2)C1] | 0.99 | 66 |
| 29 | 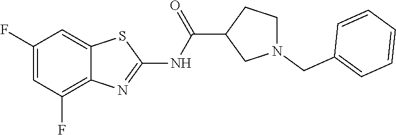 [SMILES: O=C(Nc1nc2c(F)cc(F)cc2s1)C1CCN(Cc2ccccc2)C1] | n/a | n/a |
| 30 | 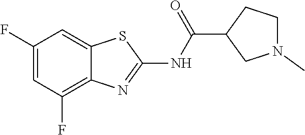 [SMILES: CN1CCC(C(=O)Nc2nc3c(F)cc(F)cc3s2)C1] | 1.79 | 84 |
| 31 | 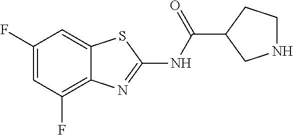 [SMILES: O=C(Nc1nc2c(F)cc(F)cc2s1)C1CCNC1] | 1.57 | 84 |
| 32 | 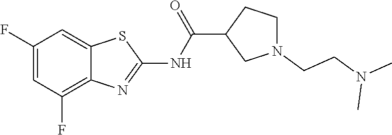 [SMILES: CN(C)CCN1CCC(C(=O)Nc2nc3c(F)cc(F)cc3s2)C1] | 1.63 | 81 |

### Example 5

### Bioactivity of Compound 19

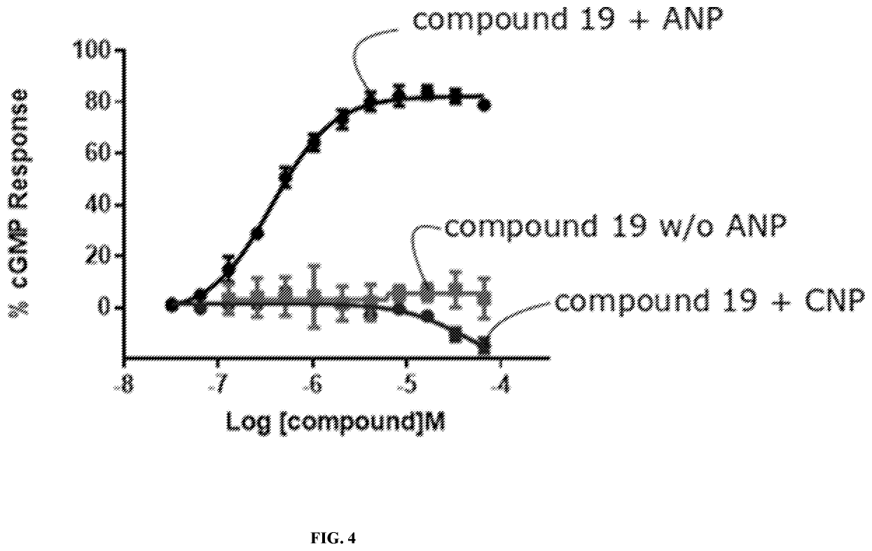

Compound 19 as a pGC-A selective, positive allosteric modulator exhibited good potency (EC₅₀=0.39 μM) and was devoid of cGMP activity in cells expressing pGC-B in the presence of CNP and in cells expressing pGC-A in the absence of ANP (See FIG. 4).

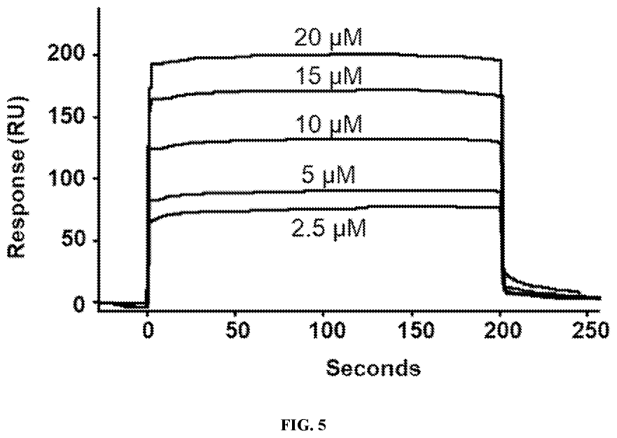

Binding Studies of compound 19 to Human pGC-A Receptor: Surface plasmon resonance (SPR) binding analysis was performed by employing the highly sensitive Biosensing Instrument 4500 system for small molecule binding. For these studies the extracellular domain of human pGC-A was used together with compound 19 at increasing concentrations. As illustrated in FIG. 5, compound 19 binding to human pGC-A was clearly validated with increasing Response (RU) signal with increasing concentration of compound 19.

### Example 6

### Pharmacokinetics and Bioavailability of Compound 19

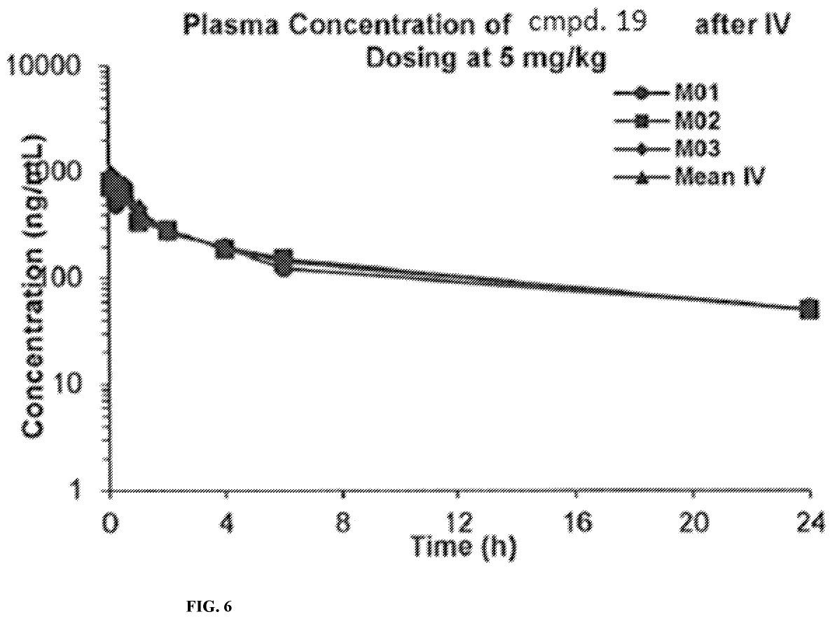

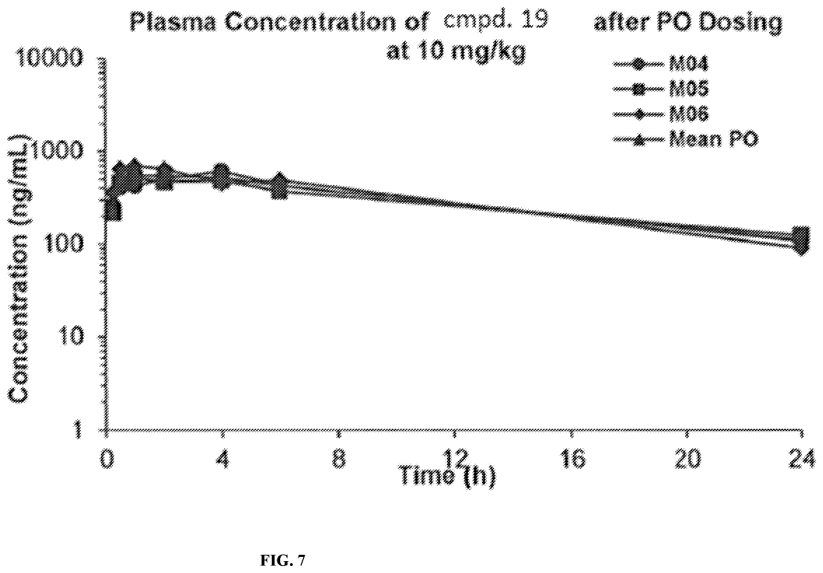

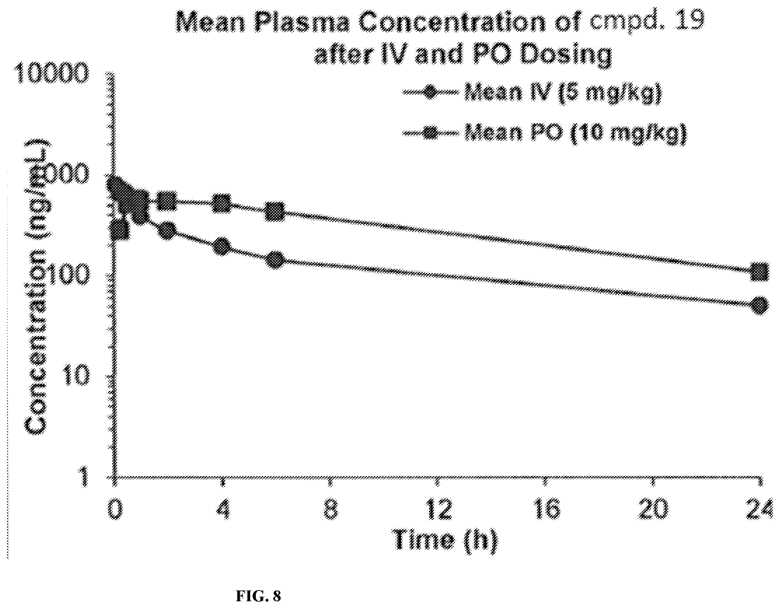

Plasma concentration of compound 19 after IV and/or PO dosing is shown in FIGS. 6-8. Profile of compound 19 absorption, distribution, metabolism and elimination is shown in Table 4.

TABLE 4

| | Value for |
|---|---|
| Property | cmpd. 19 |
| Polar Surface Area (A², Calculated by ChemBioDraw) | 47.94 |
| CLogP unionized | 3.57 |
| LogD (pH 7.4) | 3.04 |
| Plasma Stability (% remaining at 1 h) Human/Mouse | 107.4/109.7 |
| Hepatic Microsomal Stability | 98.2/83.6 |
| (% remaining at 1 h) Human/Mouse | |

| | | |
|---|---|---|
| Bioavailability Mean | Cₘₐₓ (ng/ml) | 605 |
| PO (Mouse) | T_{1/2} (h) | 9.13 |
| | Exposure (AUC ng h/mL) | 7095 |
| | Oral Bioavailability (%) | 107 |

| | |
|---|---|
| Clearance Mean IV (mouse mL/min/kg) | 20.3 |
| Vdₛₛ Mean IV (Mouse L/Kg) | 16.8 |

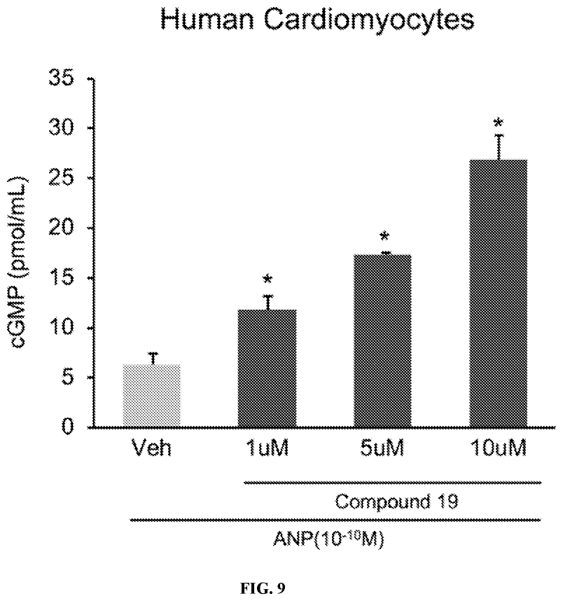

Cyclic GMP Activity in Human Cardiomyocytes: FIG. 9 shows cGMP dose response to increasing doses of compound 19 in the presence of ANP (10⁻¹⁰ M) in primary human cardiomyocytes (HCMs) which have 10-100 fold less pGC-A expression than HEK 293 pGC-A cells. Notably, compound 19 at similar dose alone did not generate cGMP generation (data not illustrated). This clear increase in cGMP generation supports the concept that compound 19 is pGC-A positive allosteric modulator that has the potential to mediate protection in HCMs.

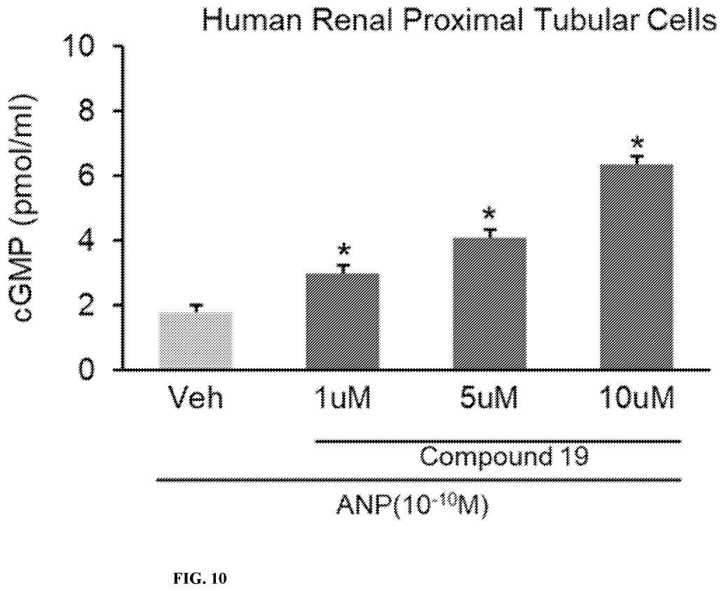

Cyclic GMP Activity in Human Renal Proximal Tubular Cells: FIG. 10 shows cGMP dose response to increasing doses of compound 19 in the presence of ANP (10⁻¹⁰ M) in primary human renal proximal tubular cells (HRPTCs) which have 10-100 fold less pGC-A expression than HEK 293 pGC-A cells. HRPTCs represent an important human cell line as renal tubular injury and apoptosis leads to impaired renal function and structure. Notably, compound 19 at similar dose alone did not generate cGMP generation (data not illustrated). This clear increase in cGMP generation supports the concept that compound 19 is pGC-A positive allosteric modulator that has the potential to mediate protection in HRPTCs.

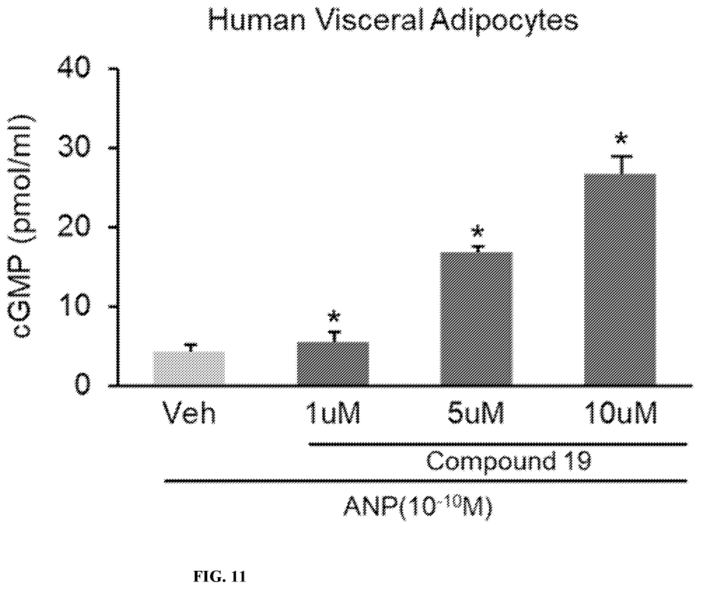

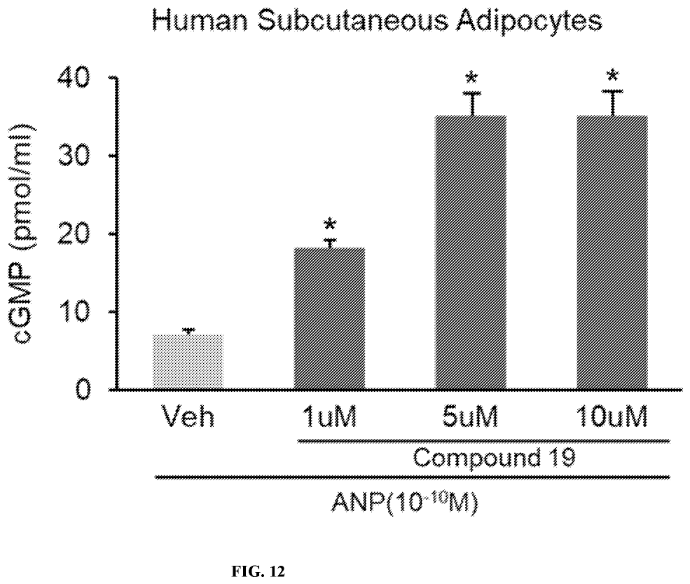

Cyclic GMP Activity of compound 19 in Human Adipocytes: dose response cGMP response to increasing doses of compound in the presence of ANP (10⁻¹⁰ M) was performed in primary human adipocytes (HAs; visceral and subcutaneous), which have ˜100 fold less pGC-A expression than HEK 293 pGC-A cells. Illustrated in FIGS. 11 and 12 is the cGMP generation in HAs to increasing concentrations of compound 19 (1, 5 and 10 μM) in the presence of ANP (10⁻¹⁰ M). Notably, compound 19 at similar dose alone did not generate cGMP generation (data not illustrated). This clear increase in cGMP generation supports the conclusion that compound 19 is pGC-A PAM that mediates protection in HAs.

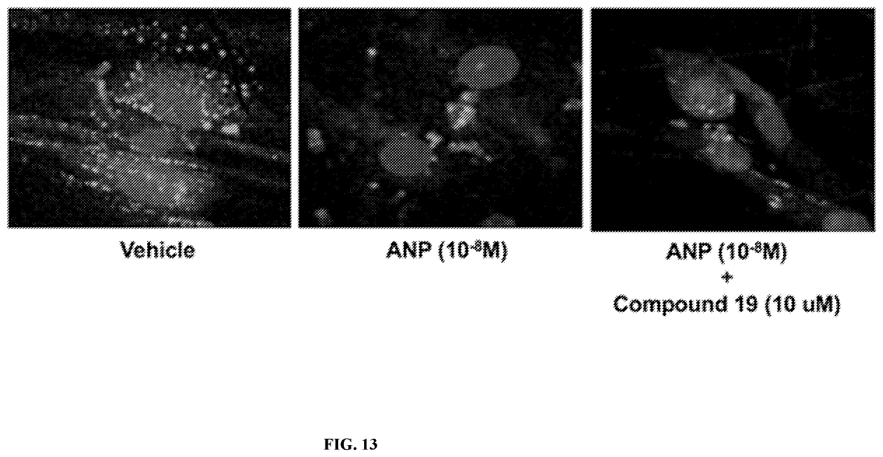

Studies of compound 19 augmenting browning and glycerol production in Human Visceral Adipocytes to ANP: human visceral adipocytes were isolated and cultured to assess the potentiation of browning and glycerol production by compound 19 mediated by ANP (FIG. 13). Staining revealed diffuse lipid droplets in vehicle treated adipocytes while ANP reduced lipid droplets over a 48 hour period of treatment. Addition of compound 19 augmented expression of the browning protein UCP1 consistent with metabolic action of compound 19. While not illustrated, compound 19 also dose dependently increased ANP induction of glycerol in human visceral adipocytes.

### Example 7

### GC-A Binding

Methods (GC-A Binding Studies): Surface plasmon resonance (SPR) measurements were performed at 25° C. on a BI-4500 SPR instrument (Biosensing Instrument Inc. Tempe AZ). As per the instructions by the Biosensing instrument manual, 400 mM nickel sulfate in de ionized water was linked to the Ni-NTA sensor chip (Biosensing Instrument Inc. Tempe Ariz. Then 40 μg/ml of extracellular domain human GC-A recombinant protein (MyBioSource, Inc. San Diego, Calif.), containing 12 histidine residues on the C-terminus, was then immobilized to the nickel sulfate on the Ni-NTA sensor chip. (Note: GC-A receptor in this study was tagged differently when compared to the receptor used in the binding study described in Example 5; hence, the numerical results differ; however, the interpretation of data obtained in this example remains congruent with the data obtained in Example 5). After, the chip was washed with buffer (150 mM NaCl, 50 μM EDTA pH 7.4, 0.1% DMSO), then 150 μL of sequentially diluted Compound 19 (0.625, 1.25, 2.5, 5, 10 04) alone was injected at the rate of 60 ul/min and allowed to dissociate for 60 seconds, or, 150 μL of sequentially diluted ANP (0.31, 0.625, 1.25, 2.5 and 5 nM) alone, or ANP (0.16, 0.31, 0.625, 1.25 and 2.5 nM) with Compound 19 (10 04) was injected at the rate of 60 μL/min and allowed to dissociate for 200 seconds. Data was collected as sensorgrams. Binding kinetics were derived from sensorgrams using BI-Data Analysis Program (Biosensing Instrument, Tempe Ariz.). Two series of binding studies were performed with increasing concentrations of Compound 19 alone, ANP alone or increasing concentrations of ANP with a fixed dose of Compound 19.

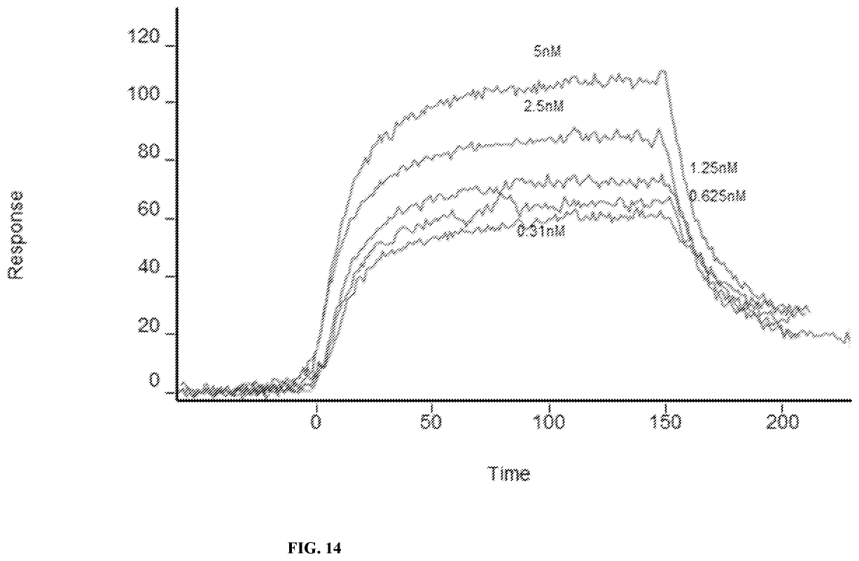

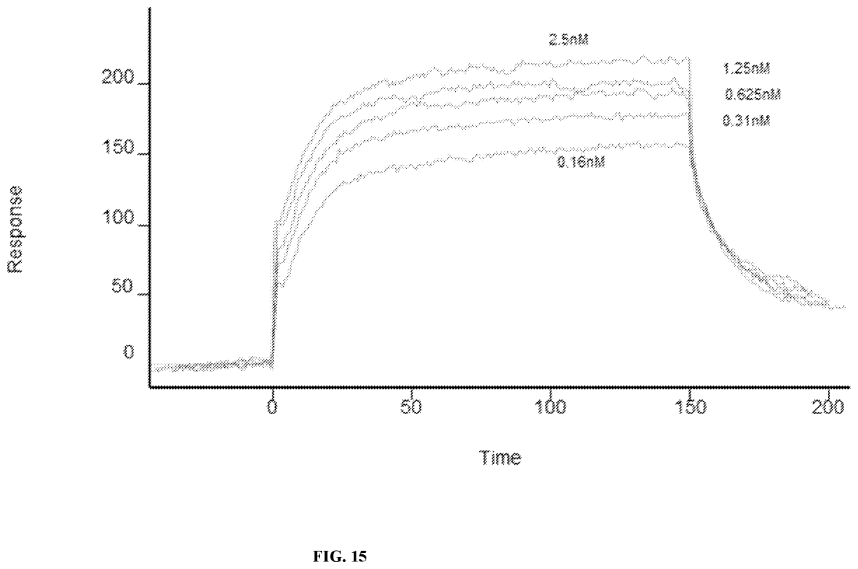

Results: State of the art SPR analysis was performed by employing the highly sensitive Biosensing Instrument 4500 system for binding of Compound 19 or ANP alone and MCUF-651 in the presence of increasing concentrations of ANP to the extracellular domain of human GC-A. With Compound 19 alone, the binding of MCUF-651 to human GC-A was confirmed, with a K_{D} of 390000 pM. Strong binding of ANP at increasing concentrations to human GC-A was validated with a K_{D} of 715 pM (FIG. 14). Importantly, the K_{D} was shifted lower to 58 pM in the presence of a fixed dose (10 μM) of Compound 19 and increasing concentrations of ANP (FIG. 15).

### Example 8

### Inhibition of Human Cardiomyocyte Hypertrophy with Compound 19

Methods (Inhibition of Human Cardiac Hypertrophy In Vitro): primary human cardiomyocytes (HCMs) were grown in growth media to 50% confluence and then were subjected to serum starvation for 24 hrs. After that, 5 ng/mL of the cytokine TGFβ-1 (R&D Systems) was added to the media for 48 hrs to induce HCM hypertrophy. Then, cells were treated with 10⁻¹⁰ or 10⁻⁸ M of ANP alone or in the presence of Compound 19 at doses of 1 μM, 5 μM and 10 μM for another 48 hrs. Normal cell buffer served as a vehicle only control, where TGFP-1 alone served as the positive control. Analysis for cell surface area (μm²) was performed using the ImageJ software at the end of the study.

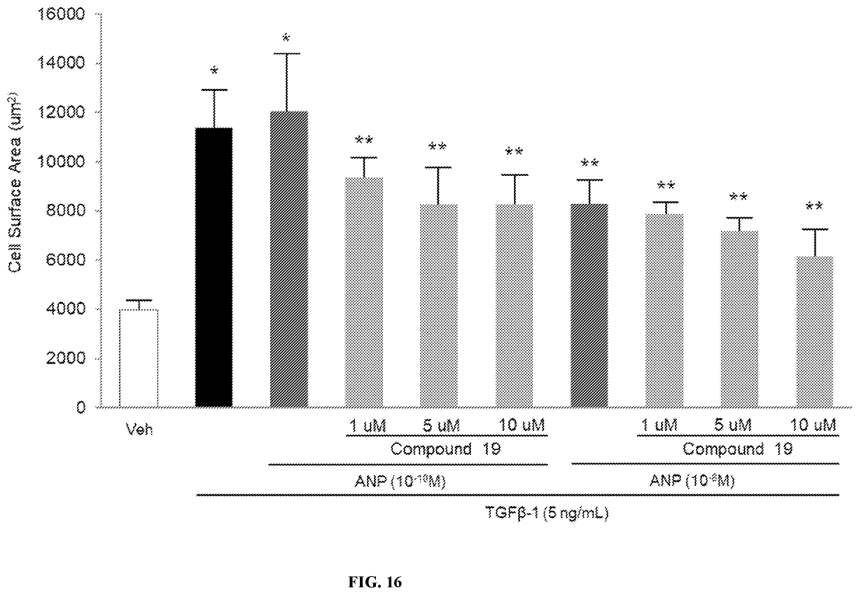

Results: to further support the cGMP generation in human primary cells leads to favorable biological GC-A mediated, the anti-hypertrophic effects were assessed of Compound 19 on live HCMs stressed with TGF-β1 (5 ng/mL), using a state-of-the-art live cell imaging platform (IncuCyte System). As illustrated in FIG. 16, 96 hour exposure to TGFβ-1 resulted in a significant increase in HCM hypertrophy. Treatment with ANP (10⁻¹⁰ M) alone had no effect in the inhibition of HCM hypertrophy. Notably, treatment with ANP (10⁻¹⁰ M) in the presence of Compound 19, ANP (10⁻⁸ M) alone as well as ANP (10⁻⁸ M) in the presence of Compound 19 resulted in a significant inhibition of HCM hypertrophy.

### Example 9

### In Vivo Actions of Compound 19 in Spontaneously Hypertensive Rats (SHRs)

Methods (IV Bolus Administration of Compound 19 in (SHRs): the cGMP generating and blood pressure lowering actions of a single IV bolus dose of Compound 19 at 10 mg/kg in spontaneously hypertensive rats (SHRs; n=2; 12 weeks old; Envigo, East Millstone, N.J.) were investigated. Studies were performed in accordance with the Animal Welfare Act and with approval of the Mayo Clinic Institutional Animal Care and Use Committee.

SHRs were anesthetized in an induction chamber with inhaled isoflurane 3%.

During this brief state of anesthesia, SHRs were administered Inactin (100 mg/kg; IP to induce anesthesia and then maintained with by additional Inactin 100 mg/kg (IP), as required. Once adequate anesthesia was achieved, the anesthetized SHRs were subjected to vessel and bladder cannulation for Compound 19 administration, blood sampling and urine collection. A polyethylene (PE)-50 tube catheter was placed into the jugular vein for Compound 19 intravenous (IV) administration. The carotid artery was cannulated with a PE-50 tube catheter for blood pressure monitoring and blood sampling/collection. The bladder was accessed and cannulated with a PE-50 tube catheter for passive urine collection. After completion of the above procedural set up, a 45-minute equilibration period was performed that included a 30-minute baseline urine collection and a single baseline blood sample was performed. After which, a single IV bolus of Compound 19 at a dose of 10 mg/kg was administered, followed by a 60-minute clearance to collect urine and blood samples. At the end of the 60-minute clearance, the anesthetized rats were euthanized by exsanguination and all blood and urine samples were measured to determine plasma and urinary cGMP levels using a cGMP ELISA kit (Enzo Life Sciences, Farmingdale, N.Y.) as instructed by the manufacturer.

Results: To further the therapeutic potential of GC-A stimulation via small molecule positive allosteric modulation, we performed an acute study to evaluate

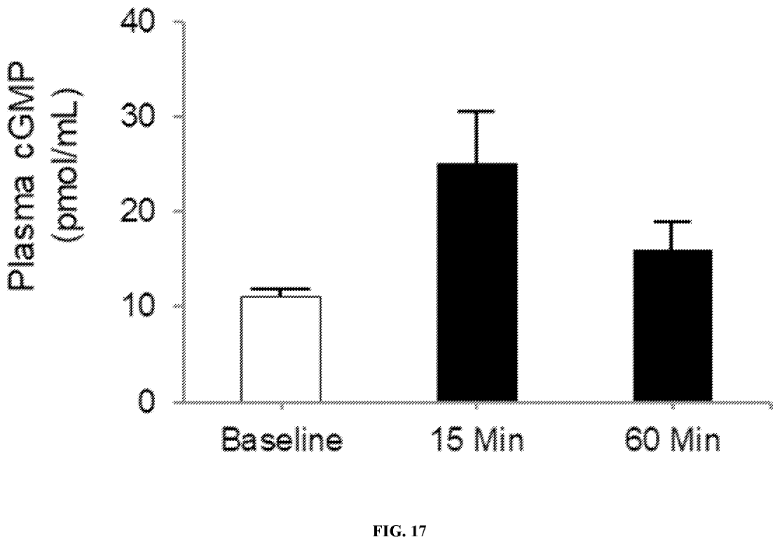

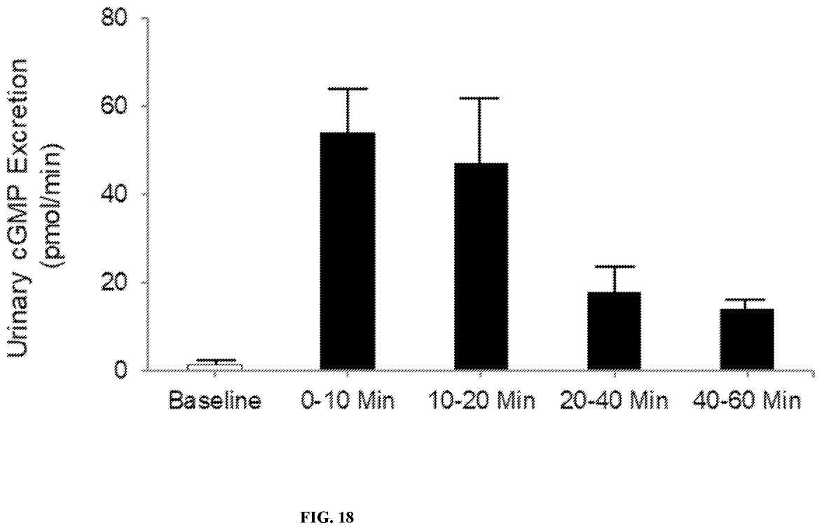

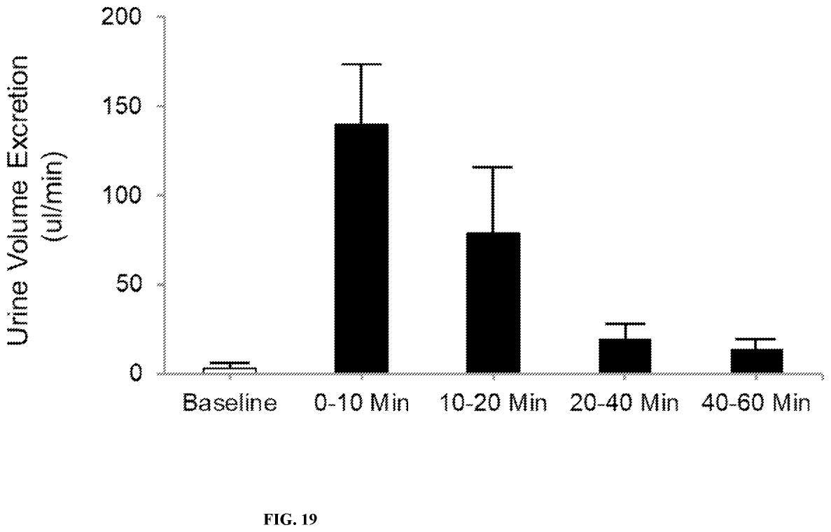

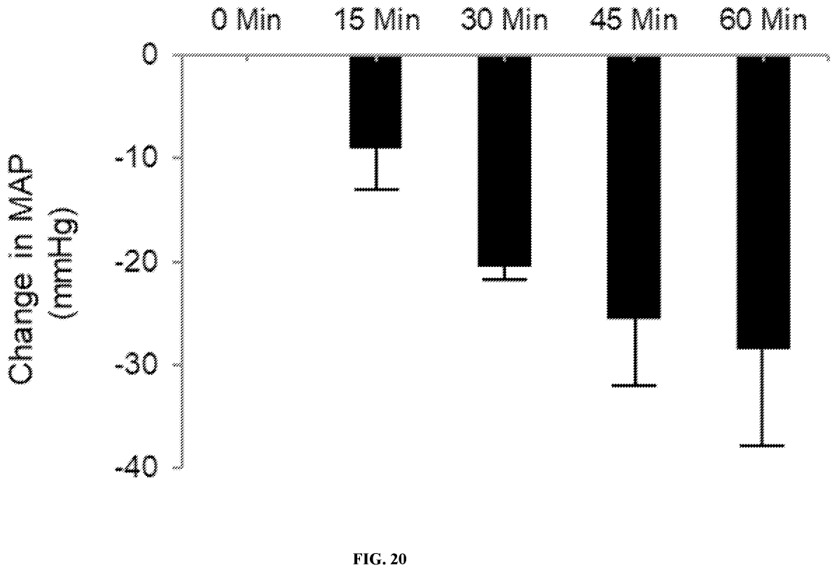

Compound 19 in SHRs to determine its ability to elevate plasma and urinary cGMP in vivo consistent with target engagement. A single IV bolus of Compound 19 at a dose of 10 mg/kg produced an increase in plasma (FIG. 17) and urinary (FIG. 18) cGMP, compared to baseline (pre-Compound 19 administration) thus consistent with potentiating endogenous circulating ANP and BNP at GC-A as predicted from our in vitro studies. Furthermore, these elevations in cGMP with Compound 19 resulted in an increase in diuresis (FIG. 19) and a decrease in mean arterial pressure (FIG. 20).

### Example 10

### Ex Vivo Compound 19 Therapeutic Potency in Human Plasma from Normal Subjects and Patients with Hypertension and Heart Failure

Methods: Stored human plasma samples from normal subjects and patients with hypertension and HF were utilized. The details of the recruitment of these participants are previously reported. All participants gave written informed consent and the Institutional Review Board (IRB) at Mayo Clinic approved this study. From all cohorts, plasma ANP was determined by a Mayo developed ANP radioimmunoassay, while plasma BNP was measured using a 2-site immunoenzymatic sandwich assay (Biosite Inc, Alere, France). HEK293 overexpressing human GC-A were cultured and grown as described above. Cells were grown in 48-well plates to 80-90% confluence. On the day of the experiment, cells were pre-incubated with Compound 19 at doses of 1, 5 or 10 μM and without (Veh) in 250 μL treatment buffer for 5 min at 37° C. 25 μL of human plasma was then added and incubated for additional 10 min. After, cells were washed with PBS once, lysed with 0.1M HCl and intracellular cGMP levels were measured in the lysate using a commercial cGMP ELISA kit (Enzo Life Sciences, Farmingdale, N.Y.) as instructed by the manufacturer.

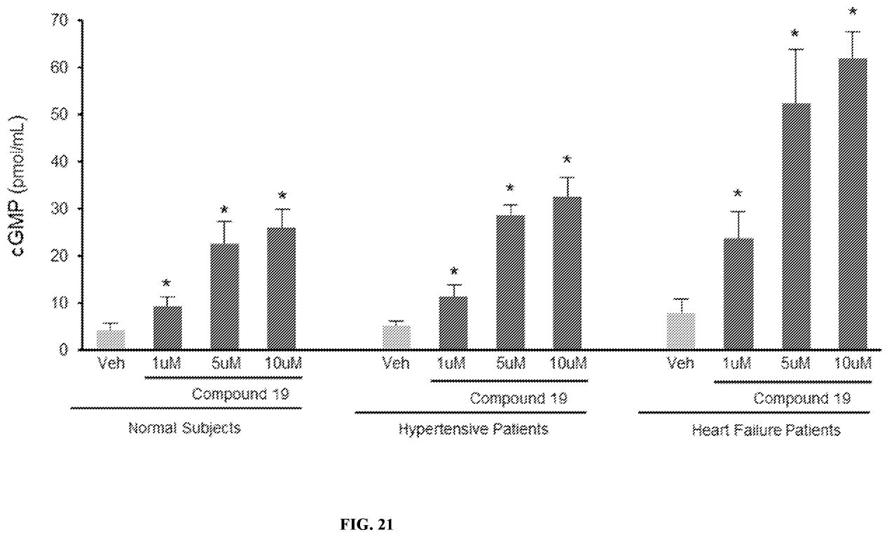

Results: To further define the therapeutic potential of Compound 19, a novel ex vivo assay was developed in which human plasma was utilized from normal subjects, and patients with hypertension and HF (n=6 per group) which have various levels of circulating ANP and BNP, the endogenous ligands of GC-A. Table 5 reports subject characteristics and their respective plasma ANP and BNP levels. As illustrated in FIG. 21, Compound 19 potentiated the generation of cGMP levels in HEK293 GC-A cells in all three cohorts. Importantly, Compound 19 demonstrated greatest cGMP potency in HF plasma in which ANP and BNP circulating levels were the highest. Importantly, these ex vivo findings provide validation that Compound 19 possesses GC-A enhancing action in human plasma and operates in a PAM mode with increasing cGMP generation in association with increasing concentrations of endogenous ANP and BNP.

TABLE 5
Human Subject Characteristics

| | Healthy | Hypertensive | Heart Failure |
|---|---|---|---|
| | (n = 6) | (n = 6) | (n = 6) |
| Age, years | 59 ± 8 | 69 ± 9 | 65 ± 10 |
| Sex, female (%) | 75% | 50% | 75% |
| BMI, kg/m² | 28 ± 3 | 25 ± 1 | 29 ± 5 |
| eGFR, mL/min/1.73 m² | 75 ± 15 | 70 ± 8 | 50 ± 25 |
| ANP, pg/mL | 26 ± 12 | 13 ± 8 | 350 ± 140 |
| BNP, pg/mL | 27 ± 14 | 59 ± 37 | 1226 ± 711 |

Values are presented as mean ± SD, n (%).
BMI = body mass index;
eGFR = estimated glomerular filtration rate;
ANP = atrial natriuretic peptide;
BNP = b-type natriuretic peptide

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1-45. (canceled)

46. A compound of Formula (I): 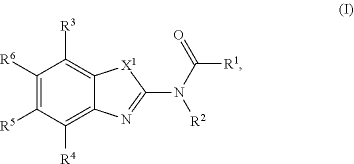
or a pharmaceutically acceptable salt thereof, wherein:
X¹ is selected from S, O, and NR²;
R¹ is selected from any one of the following groups: 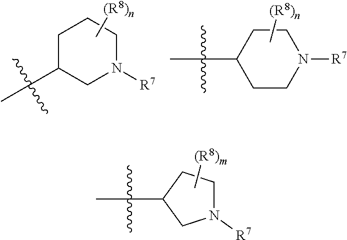
R² is selected from H and C₁₋₃ alkyl;
R³ and R⁵ are each independently selected from H, halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} and S(O)₂NR^{c1}R^{d1};
R⁴ is halo;
R⁶ is selected from H and halo;
each R⁷ is independently selected from H, C₁₋₆ alkyl, C₁₋₆ haloalkyl, C₂₋₆ alkenyl, C₂₋₆ alkynyl, C(O)R^{b1}, C(O)NR^{c1}R^{d1}, C(O)OR^{a1}, S(O)₂R^{b1}, and S(O)₂NR^{c1}R^{d1}, wherein said C₁₋₆ alkyl, C₂₋₆ alkenyl, and C₂₋₆ alkynyl are each optionally substituted with 1, 2 or 3 substituents independently selected from Cy¹, halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each Cy¹ is independently selected from C₆₋₁₀ aryl and C₃₋₁₀ cycloalkyl, each of which is optionally substituted with 1, 2, 3, 4, or 5 substituents independently selected from R^{Cy1};
each R^{Cy1} is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each R⁸ is independently selected from halo, CN, NO₂, C₁₋₆ alkyl, C₁₋₆ haloalkyl, C₂₋₆ alkenyl, C₂₋₆ alkynyl, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl, C₂₋₆ alkenyl, and C₂₋₆ alkynyl are each optionally substituted with 1, 2 or 3 substituents independently selected from halo, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1} and S(O)₂NR^{c1}R^{d1};
each n is independently 0, 1, 2, 3, 4, 5, 6, 7, or 8;
m is 0, 1, 2, 3, 4, 5, or 6;
each R^{a1} and R^{b1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene, wherein said C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene are optionally substituted with 1, 2, 3, 4, or 5 substituents independently selected from R^{g};
each R^{c1} and R^{d1} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, C₃₋₁₀ cycloalkyl-C₁₋₄ alkylene, C(O)R^{b2}, C(O)NR^{c2}R^{d2}, C(O)OR^{a2}, NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene is optionally substituted with 1, 2, 3, 4, or 5 substituents independently selected from R^{g};
each R^{a2}, R^{b2}, R^{c2}, and R^{d2} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, C₃₋₁₀ cycloalkyl-C₁₋₄ alkylene, and R^{g}, wherein said C₁₋₆ alkyl, C₂₋₆ alkenyl, C₂₋₆ alkynyl, C₆₋₁₀ aryl, C₃₋₁₀ cycloalkyl, C₆₋₁₀ aryl-C₁₋₄ alkylene, and C₃₋₁₀ cycloalkyl-C₁₋₄ alkylene is optionally substituted with 1, 2, 3, 4, or 5 substituents independently selected from R^{g};
or any R^{c1} and R^{d1} together with the N atom to which they are attached form a 4-, 5-, 6-, or 7-membered heterocycloalkyl group optionally substituted with 1, 2, or 3 substituents independently selected from R^{g};
or any R^{c2} and R^{d2} together with the N atom to which they are attached form a 4-, 5-, 6-, or 7-membered heterocycloalkyl group optionally substituted with 1, 2, or 3 substituents independently selected from R^{g};
each R^{e1} is independently selected from H, C₁₋₄ alkyl, C₁₋₄ alkoxy, OH, and CN; an
each R^{g} is independently selected from OH, NO₂, CN, halo, C₁₋₆ alkyl, C₂₋₆ alkenyl, C₂₋₆ alkynyl, C₁₋₄ haloalkyl, C₁₋₆ alkoxy, C₁₋₆ haloalkoxy, cyano-C₁₋₃ alkylene, HO—C₁₋₃ alkylene, amino, C₁₋₆ alkylamino, di(C₁₋₆ alkyl)amino, (C₃₋₁₀ cycloalkyl)amino, di(C₃₋₁₀ cycloalkyl)amino, thio, C₁₋₆ alkylthio, C₁₋₆ alkylsulfinyl, C₁₋₆ alkylsulfonyl, carbamyl, C₁₋₆ alkylcarbamyl, di(C₁₋₆ alkyl)carbamyl, carboxy, C₁₋₆ alkylcarbonyl, C₁₋₆ alkoxycarbonyl, C₁₋₆ alkylcarbonylamino, C₁₋₆ alkylsulfonylamino, aminosulfonyl, C₁₋₆ alkylaminosulfonyl, di(C₁₋₆ alkyl)aminosulfonyl, aminosulfonylamino, C₁₋₆ alkylaminosulfonylamino, di(C₁₋₆ alkyl)aminosulfonylamino, aminocarbonylamino, C₁₋₆ alkylaminocarbonylamino, and di(C₁₋₆ alkyl)aminocarbonylamino.

47. The compound of claim 46, wherein the compound of Formula (I) has formula: 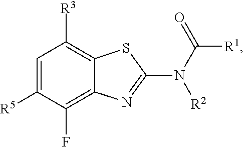
or a pharmaceutically acceptable salt thereof.

48. The compound of claim 46, wherein the compound of Formula (I) has formula: 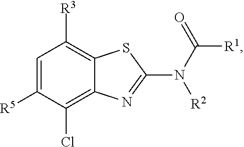
or a pharmaceutically acceptable salt thereof.

49. The compound of claim 46, wherein the compound of Formula (I) has formula: 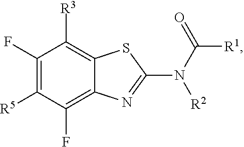
or a pharmaceutically acceptable salt thereof.

50. The compound of claim 46, wherein:
R³ and R⁵ are each independently selected from H, halo, CN, NO₂, C₁₋₆ alkyl, C₁₋₆ haloalkyl, OR^{a1}, wherein said C₁₋₆ alkyl is optionally substituted with CN, NO₂, OR^{a1}, C(O)R^{b1}, C(O)NR^{c1}R^{d1}, C(O)OR^{a1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} or S(O)₂NR^{c1}R^{d1};
each R⁸ is independently selected from halo, CN, NO₂, C₁₋₆ alkyl, C₁₋₆ haloalkyl, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1}, and S(O)₂NR^{c1}R^{d1}; wherein said C₁₋₆ alkyl is optionally substituted with halo, CN, NO₂, OR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)₂R^{b1}, NR^{c1}S(O)₂NR^{c1}R^{d1}, S(O)₂R^{b1} or S(O)₂NR^{c1}R^{d1};
each n is independently 1 or 2; and
m is 1 or 2.

51. The compound of claim 46, wherein:
R³ and R⁵ are each independently selected from H, halo, C₁₋₆ alkyl, C₁₋₆ haloalkyl, C₁₋₆ alkoxy, and C₁₋₆ haloalkoxy;
each R⁸ is independently selected from halo, C₁₋₆ alkyl, C₁₋₆ haloalkyl, C₁₋₆ alkoxy, and C₁₋₆ haloalkoxy;
R² is H;
each n is independently 1 or 2; and
m is 1 or2.

52. The compound of claim 46, wherein:
R³ and R⁵ are each H;
R² is H;
each n is 0;
and m is 0.

53. The compound of claim 46, wherein R¹ is:

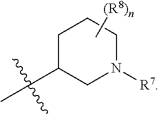

54. The compound of claim 46, wherein R¹ is:

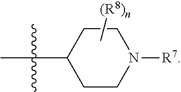

55. The compound of claim 46, wherein R¹ is:

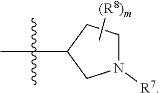

56. The compound of claim 46, wherein the compound of Formula (I) has formula: 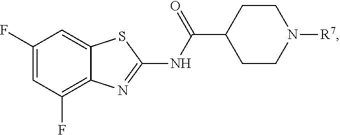
or a pharmaceutically acceptable salt thereof.

57. The compound of claim 46, wherein the compound of Formula (I) has formula: 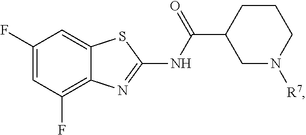
or a pharmaceutically acceptable salt thereof.

58. The compound of claim 46, wherein the compound of Formula (I) has formula: 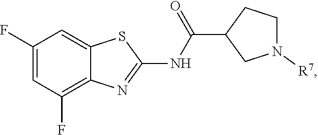
or a pharmaceutically acceptable salt thereof.

59. The compound of claim 46, wherein R⁷ is selected from H, C₁₋₆ alkyl, C(O)R^{b1}, C(O)OR^{a1}, and S(O)₂R^{b1}, wherein said C₁₋₆ alkyl is optionally substituted with C₆₋₁₀ aryl or NR^{c1}R^{d1}.

60. The compound of claim 46, wherein the compound of Formula (I) is selected from any one of the following compounds: 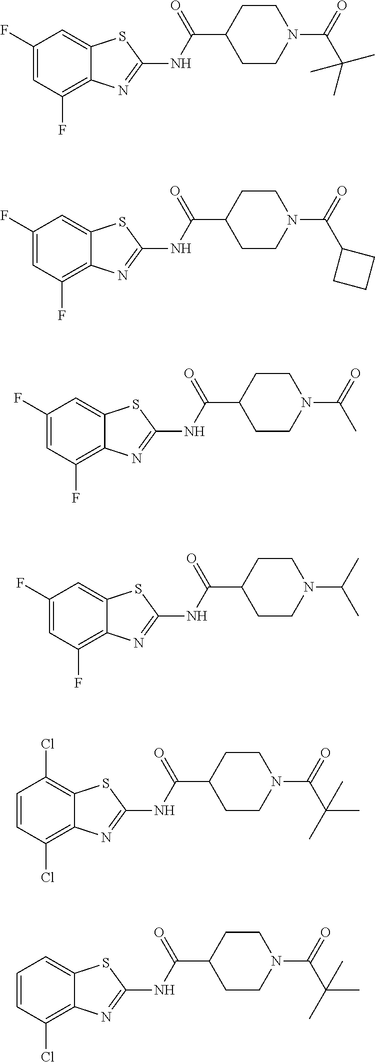 [SMILES: CC(C)(C)C(=O)N1CCC(C(=O)Nc2nc3c(F)cc(F)cc3s2)CC1 ; O=C(Nc1nc2c(F)cc(F)cc2s1)C1CCN(C(=O)C2CCC2)CC1 ; CC(=O)N1CCC(C(=O)Nc2nc3c(F)cc(F)cc3s2)CC1 ; CC(C)N1CCC(C(=O)Nc2nc3c(F)cc(F)cc3s2)CC1 ; CC(C)(C)C(=O)N1CCC(C(=O)Nc2nc3c(Cl)ccc(Cl)c3s2)CC1 ; CC(C)(C)C(=O)N1CCC(C(=O)Nc2nc3c(Cl)cccc3s2)CC1]
or a pharmaceutically acceptable salt thereof.

61. The compound of claim 46, wherein the compound of Formula (I) is selected from any one of the following compounds: 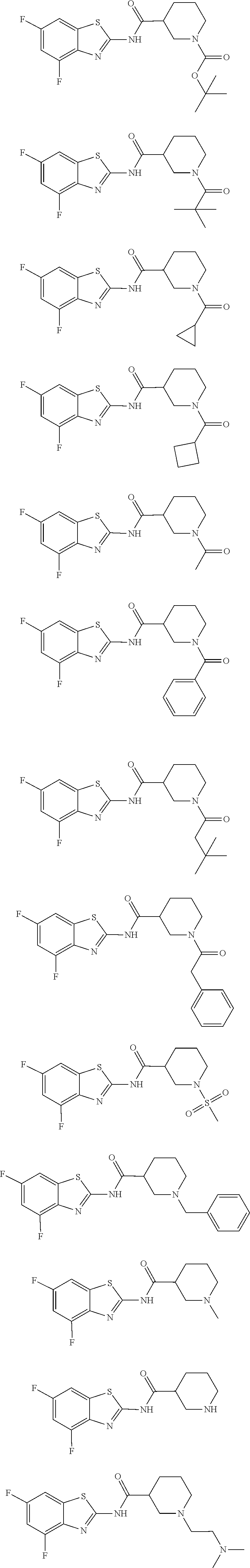
or a pharmaceutically acceptable salt thereof.

62. The compound of claim 46, wherein the compound of Formula (I) is selected from any one of the following compounds: 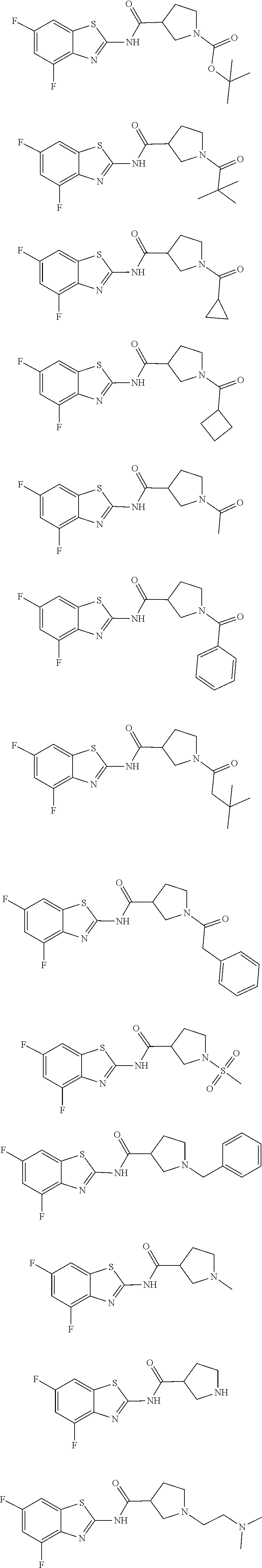
or a pharmaceutically acceptable salt thereof.

63. A pharmaceutical composition comprising a compound of claim 46, or a pharmaceutically acceptable salt thereof, and a pharmaceutically acceptable carrier.

64. A method of modulating particulate guanylyl cyclase receptor A (pGC-A) in a cell, the method comprising contacting the cell with an effective amount of the compound of claim 46, or a pharmaceutically acceptable salt thereof.

65. A method of treating a disease or condition responsive to modulation of a particulate guanylyl cyclase receptor A (pGC-A) in a subject, the method comprising administering to the subject in need thereof a therapeutically effective amount of the compound of claim 46, or a pharmaceutically acceptable salt thereof.
